(19) United States
(12) Patent Application Publication
(10) Pub. No.: US 2012/0142682 A1
(43) Pub. Date: Jun. 7, 2012

(54) ANTIVIRULENCE COMPOUNDS INHIBITING BACTERIAL MONO-ADP-RIBOSYLTRANSFERASE TOXINS

(75) Inventor: Allen Rod Merrill, Ariss (CA)

(73) Assignee: UNIVERSITY OF GUELPH, Guelph (CA)

(21) Appl. No.: 13/310,051

(22) Filed: Dec. 2, 2011

Related U.S. Application Data
(60) Provisional application No. 61/418,917, filed on Dec. 2, 2010.

(51) Int. Cl. A61K 31/381; A61P 31/04; A61K 31/353; A61K 31/496; A61K 31/5377

(52) U.S. Cl. 514/233.5; 514/443; 514/254.11; 514/456

(57) ABSTRACT

Compounds that inhibit bacterial virulence factors from the mono-ADP-ribosyltransferase (mART) family of toxins have been identified that are not toxic to cells or the producing bacterial pathogen. These compounds have great potential as antivirulence agents for treating many bacterial infections and disease states.

### FIELD OF THE APPLICATION

The present application is in the field of antivirulence compounds, or compounds that are inhibitors of bacterial virulence factors.

### BACKGROUND

The mono-ADP-ribosyltransferase (mART) family is a group of toxic bacterial enzymes or bacterial virulence factors. The best-characterized and well-known members of this family are cholera toxin (CT) from Vibrio cholerae, diphtheria toxin (DT) from Corynebacterium diphtheriae, pertussis toxin from Bordella pertussis, heat-labile enterotoxin from Escherichia coli, C3-like exoenzyme produced by Clostridium botulinum and Clostridium limosum, and exotoxin A (ExoA) from Pseudomonas aeruginosa.

These bacterial virulence factors contribute to many disease states in plants, animals and humans. For example, cholera, caused by an infection by V. cholerae, affects 3-5 million people and causes 100,000-130,000 deaths a year as of 2010. Primary treatment is with oral rehydration solution and, in some cases, intravenous fluids and antibiotics in those with severe disease state. As another example, P. aeruginosa infects primarily immunocompromised individuals through the contact of these individuals with contaminated surfaces found on and in medical equipment or infected individuals. P. aeruginosa is naturally resistant to a large range of antibiotics and may demonstrate additional resistance after unsuccessful treatment. The use of conventional antibiotics presents several shortcomings such as acting against an essential metabolic factor, inducing tolerance or drug-resistance mutations in the infecting bacteria, producing undesirable side-effects.

The mechanism of action of the mART enzymes is known. They act on NAD+ and facilitate the scission of the glycosidic bond (C—N) between nicotinamide and its conjugated ribose followed by the transfer of the ADP-ribose group to a nucleophilic residue on a target macromolecule ¹². Therefore, these toxins function as enzymes that target various host proteins and covalently attach an ADP-ribose moiety that alters host protein function.

In light of the above, it follows that one strategy to combat the effects of bacterial infection is to inhibit the action of these virulence factors. It is now generally appreciated that an antivirulence approach is a powerful alternative strategy to antibacterial treatment and vaccine development¹⁰ and that it may require multiple tactics to resolve the current drug resistance dilemma ^{8,11}.

Hence, in light of the discussion above, there is a need for a strategy (compound, use of the compound, or method) to inhibit bacterial virulence factors which, by virtue of its design and components, would be able to overcome some of the aforementioned problems.

### SUMMARY OF THE APPLICATION

An aspect of the present application includes antivirulence compounds which inhibit bacterial virulence factors.

Antivirulence compounds offer significant advantages over conventional antibiotics since these inhibitors are directed towards specific mechanisms (targets) in the offending pathogen that promote infection rather than act against an essential metabolic factor⁹. Neutralizing the cytotoxic properties of virulence factors from microorganisms without threatening their survival offers reduced selection pressure, making it less likely to induce drug-resistant mutations⁸. Additionally, virulence-specific therapeutics avoids the undesirable effects on the host microbiota that are associated with current antibiotics.

The antivirulence compounds of the present application are inhibitors of bacterial virulence factors. In accordance with the present application, these bacterial virulence factors are from the mART family of enzymes. mART enzymes are found in CT, DT, pertussis toxin, heat-labile enterotoxin, C3-like exoenzyme, ExoA and other bacterial toxins.

In its most general embodiment, the present application includes a method to inhibit bacterial virulence factors comprising administering an effective amount of an antivirulence compound to a subject or cell in need thereof. Another embodiment of the present application is a use of an antivirulence compound to inhibit bacterial virulence factors and a use of an antivirulence compound to prepare a medicament to inhibit bacterial virulence factors.

In an embodiment, an antivirulence compound of the present application, which demonstrated a protective effect against ExoA in mammalian cells, is a compound of the Formula I:

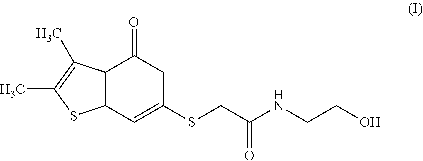

or a pharmaceutically acceptable solvate and/or prodrug thereof. Accordingly, in an embodiment of the application, there is included a method to inhibit bacterial virulence factors comprising administering an effective amount of a compound of Formula I, or a pharmaceutically acceptable solvate and/or prodrug thereof, to a subject or cell in need thereof. Another embodiment of the present application is a use of a compound of Formula I, or a pharmaceutically acceptable solvate and/or prodrug thereof, to inhibit bacterial virulence factors and a use of a compound of Formula I, or a pharmaceutically acceptable solvate and/or prodrug thereof, to prepare a medicament to inhibit bacterial virulence factors.

In an embodiment, an antivirulence compound of the present application, which demonstrated a protective effect against ExoA in mammalian cells, is a compound of the Formula II:

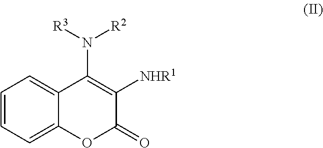

wherein:
R¹ is selected from H and C(O)C₁₋₄alkyl;
R² is H and R³ is selected from aryl and (CH₂)ₙNR⁴R⁵, or R² and R³, together with the nitrogen atom to which they are attached, form a piperidinyl, piperazinyl or morpholinyl ring;
R⁴ and R⁵ are independently selected from H, C₁₋₄alkyl or, together with the nitrogen atom to which they are attached, form a piperidinyl, piperazinyl or morpholinyl ring; and
n is 1, 2, 3 or 4,
or a pharmaceutically acceptable salt, solvate and/or prodrug thereof, to a subject or cell in need thereof.

Accordingly, in an embodiment of the application, there is included a method to inhibit bacterial virulence factors comprising administering an effective amount of a compound of Formula II, or a pharmaceutically acceptable salt, solvate and/or prodrug thereof, to a subject or cell in need thereof. Another embodiment of the present application is a use of a compound of Formula II, or a pharmaceutically acceptable salt, solvate and/or prodrug thereof, to inhibit bacterial virulence factors and a use of a compound of Formula II, or a pharmaceutically acceptable salt, solvate and/or prodrug thereof, to prepare a medicament to inhibit bacterial virulence factors.

In another embodiment of the application, there is included a method of preventing and/or treating a disease state caused by a bacterial infection comprising administering an effective amount of one or more compounds selected from a compound of Formula I and a compound of Formula II, and pharmaceutically acceptable salts, solvates and/or prodrugs thereof, to a subject or cell in need thereof. Another embodiment of the present application is a use of one or more compounds selected from a compound of Formula I, and a compound of Formula II, and pharmaceutically acceptable salts, solvates and/or prodrugs thereof, to inhibit bacterial virulence factors and a use of one or more or more compounds selected from a compound of Formula I and a compound of Formula II, and pharmaceutically acceptable salts, solvates and/or prodrugs thereof, to prepare a medicament to inhibit bacterial virulence factors. In an embodiment, the bacterial infection is, for example, a P. aeruginosa infection.

The objects and advantages of the present application will become more apparent upon reading the following non-restrictive description of the preferred embodiments thereof, given for the purpose of exemplification only, with reference to the accompanying drawings.

### BRIEF DESCRIPTION OF THE DRAWINGS

The present application will be described in greater detail with reference to the drawings in which:

FIG. 1 shows the chemical structures of several mART inhibitors. The P-series (P1-P8) inhibitors are shown along with the most active V-series inhibitor, V30, and previously characterized mART inhibitors, 1,8-naphthalimide (NAP) and N-(6-oxo-5,6-dihydrophenanthridin-2-yl)-(N,N-dimethylamino)acetamide hydrochloride (PJ34). Also shown are the structures of a NAP derivative, 4-amino-NAP, and the parent compound for PJ34, PJ97A.

FIG. 2A shows the cytoprotective effects of non-polar inhibitors in ExoA intoxicated C38 cells. (A) NAP protects cells from the toxic effects of ExoA (circles) as compared to a 0.5% DMSO control (squares). (B) Compounds PJ97A (diamonds), (C) V30 (triangles) and (D) V23 (polygons) all show varying levels of protection in C38 cells. The ExoA dose-response curve is shown in each panel as the filled squares. All compounds were assayed at a final concentration of 50 μM in the presence of 0.5% DMSO. The LD₅₀ of ExoA in C38 with 0.5% DMSO was 65±3 ng/ml. The LD₅₀ of ExoA in the presence of NAP and V30 was >1000 ng/ml, PJ97A was >750 ng/ml and V23 was 199±30 ng/ml. Experiments were performed in quadruplicates, in three independent determinations. Error bars represent the mean±the SD for each set of replicates.

FIG. 2B shows the cytoprotective effects of weak ExoA inhibitors in ExoA intoxicated C38 cells. (A) Compound V12 (triangles, (B) compound V15 (stars) and (C) compound V24 (diamonds) increased the LD₅₀ of ExoA to 228±12 ng/ml, 208±41 ng/ml and 238±16 ng/ml respectively, as compared to ExoA alone (squares), which was 65±3 ng/ml. All compounds were assayed in 0.5% DMSO. Experiments were performed in quadruplicates, in three independent determinations. Error bars represent the mean±the SD from of each set of replicates.

FIG. 3 demonstrates the effect of inhibitor treatment on the morphology of ExoA-intoxicated C38 cells. Human C38 cells were grown for 72 h (A) in the absence of ExoA and inhibitor (control), (B) 500 ng/ml ExoA, (C) 500 ng/ml ExoA and 50 μM compound V30 and (D) 500 ng/ml ExoA and 50 μM compound P1. Pictures were taken at 200× magnification with an Zeiss inverted microscope (Invertoskop 40C).

FIG. 4 shows the cytoprotective effects of water-soluble inhibitors in ExoA intoxicated C38 cells. (A) Compounds P1 (hexagons), (B) P4 (diamonds), (C)P5 (stars) and (D) P6 (triangles) protect C38 from high doses of ExoA. The ExoA dose-response curve is shown in each panel as the filled squares. All compounds were assayed at a final concentration of 50 μM. The LD₅₀ of ExoA in C38 was 61±9 ng/ml. The LD₅₀ of ExoA in the presence of compounds P1, P4, P5 and P6 was >1000 ng/ml. Experiments were performed in quadruplicates, in three independent determinations. Error bars represent the mean±the SD for each set of replicates.

FIG. 5 shows the inhibitor potency in C38 cells as determined by half maximal effective concentration (EC₅₀). C38 cells were exposed to lethal doses of ExoA (650 ng/ml, 10-times LD₅₀) at varying concentrations of each inhibitor. (A) NAP protected with an EC₅₀ of 3.8±0.9 μM and (B) V30 had an EC₅₀ value of 8.8±0.5 μM, both in the presence of 0.5% DMSO. (C) Compounds P1, (D) P4, (E) P5 and (F) P6 had EC₅₀ values of 2.9±0.8 μM, 12.6±3.3 μM, 16.7±1.9 μM and 3.4±1.6 μM respectively. EC₅₀ values represent the average value obtained from four independent determinations performed in quadruplicates. Error bars represent the mean±the SD for each set of replicates.

FIG. 6 shows the crystal structures of the catalytic fragment of cholix bound to inhibitor compounds. (A) cholix-P1, (B) cholix-P2, (C) cholix-P3, (D) cholix-P4, (E) cholix-P5, (F) cholix-P6, (G) cholix-P7, (H) cholix-P8, (I) cholix-V30, and (J) model of cholix-NAD⁺ complex. The inhibitors and the NAD⁺ substrate are shown with standard atom colors and nearby residues are shown as black sticks. Hydrogen bonds are shown in orange dashed lines. The model of the cholix-NAD⁺ complex is based upon the ExoA-NAD⁺ complex (PDB:3B78).

FIG. 7 shows the structures and chemical properties of the virtual screen inhibitors against cholix toxin. These compounds were purchased from the Chembridge Compound library (Chembridge, San Diego, Calif.) and were tested for the ability to protect both yeast and human C38 lung cells as described in Materials and Methods below. Those compounds that showed protective activity against ExoA toxin were then quantified for in vitro inhibitory activity (IC₅₀ values).

FIG. 8 shows structures and chemical properties of the directed-library inhibitors (P1-P8) and inhibitors NAP and PJ34 along with their corresponding polar derivatives. Those compounds that showed protective activity against ExoA toxin were then quantified for in vitro inhibitory activity (IC₅₀ values).

FIG. 9 shows the statistics for data collection and refinement of cholix-inhibitor structures.

FIG. 10 shows the electron density of the inhibitors bound to cholix toxin. Omit mF_{O}-DF_{C} maps around inhibitor P1, P2, P3, P4, P5, P6, P7, P8 and V30 (red, contoured at 2.5σ). The positions of the sulfur atoms present in inhibitor P8 and V30 are confirmed by the electron density of the omit map, blue, contoured at 10 and 13σ, respectively.

FIG. 11 shows the two-dimensional chemical drawings of catalytic fragment of cholix-inhibitor complexes based on the corresponding crystal structures. A) cholix-P1 (GP-D), B) cholix-P2 (GP-F), C) cholix-P3 (GP-G), D) cholix-P4 (GP-H), E) cholix-P5 (GP-I), F) cholix-P6 (GP-L), G) cholix-P7 (GP-M), H) cholix-P8 (GP-P), I) cholix-V30, and J) cholix-NAD⁺. Two-dimensional cholix-inhibitor visualization was achieved by removing inhibitor coordinates from the respective pdb file, using Open Babel (http://openbabel.sourceforge.net/) to convert these coordinates to structure data format (*.sdf) and then drawing the complex using PoseView (http://poseview.zbh.uni-hamburg.de/).

While the application will be described in conjunction with example embodiments, it will be understood that it is not intended to limit the scope of the application to such embodiments. On the contrary, it is intended to cover all alternatives, modifications and equivalents as may be included and defined by the appended claims.

### DETAILED DESCRIPTION OF THE APPLICATION

An aspect of the present application is to provide an antivirulence compound. The antivirulence compounds of the present application are inhibitors of bacterial virulence factors. Virulence factors are molecules expressed and secreted by bacteria, viruses and fungi that enable them to achieve colonisation of a niche in the host, cell adhesion, immunoevasion, immunosuppression, entry into and exit out of cells when applicable, and obtain nutrition from the host. Virulence factors are very often responsible for causing disease in the host as they inhibit certain host functions.

In accordance with this aspect of the present application, the virulence factors are bacterial virulence factors. They are also from the mART family of enzymes. The mART family of enzymes can be divided into the CT and DT groups. The CT group consists of an ExoS-like subgroup (enzymatic A-domain alone or paired with another domain) which targets the RAS family of G-proteins; the C2-like subgroup (A/B motif, where B is the translocation domain) which targets actin; the C3-like subgroup (A-only) which targets the Rho G-protein family, and the CT-PT-like subgroup (A/B5) which targets the Gα-family of G-proteins. The three characterized members of the DT group consist of three-domain A/B toxins that target the ribosomal translocase, eukaryotic elongation factor 2 ¹³. The mART family is characterized by low primary sequence identity, but the catalytic domain is structurally conserved. As mentioned above, the mechanism of action of the mART enzymes is known. They act on NAD⁺ and facilitate the scission of the glycosidic bond (C—N) between nicotinamide and its conjugated ribose followed by the transfer of the ADP-ribose group to a nucleophilic residue on a target macromolecule to ultimately alter host protein function. mART enzymes are found in CT, DT, pertussis toxin, heat-labile enterotoxin, C3-like exoenzyme, ExoA and other bacterial toxins.

An in silico approach was developed, based on fold recognition methods, to identify prospective, new mART members from bacterial genomes ¹⁴. Compounds from a virtual screen of commercially available compounds combined with a directed PARP inhibitor library were tested. PARP inhibitors are a family of enzymes which act as inhibitors of the enzyme Poly ADP ribose polymerase (PARP). They are developed for multiple indications, the most important is the treatment of cancer. The PARP family of enzymes is distinct from the mART family since the target proteins are different; however, the two families do share a common substrate: NAD. Several compounds bound tightly and inhibited toxins from P. aeruginosa and V. cholerae. The most efficacious compounds completely protected human lung epithelial cells against the cytotoxicity of these bacterial virulence factors.

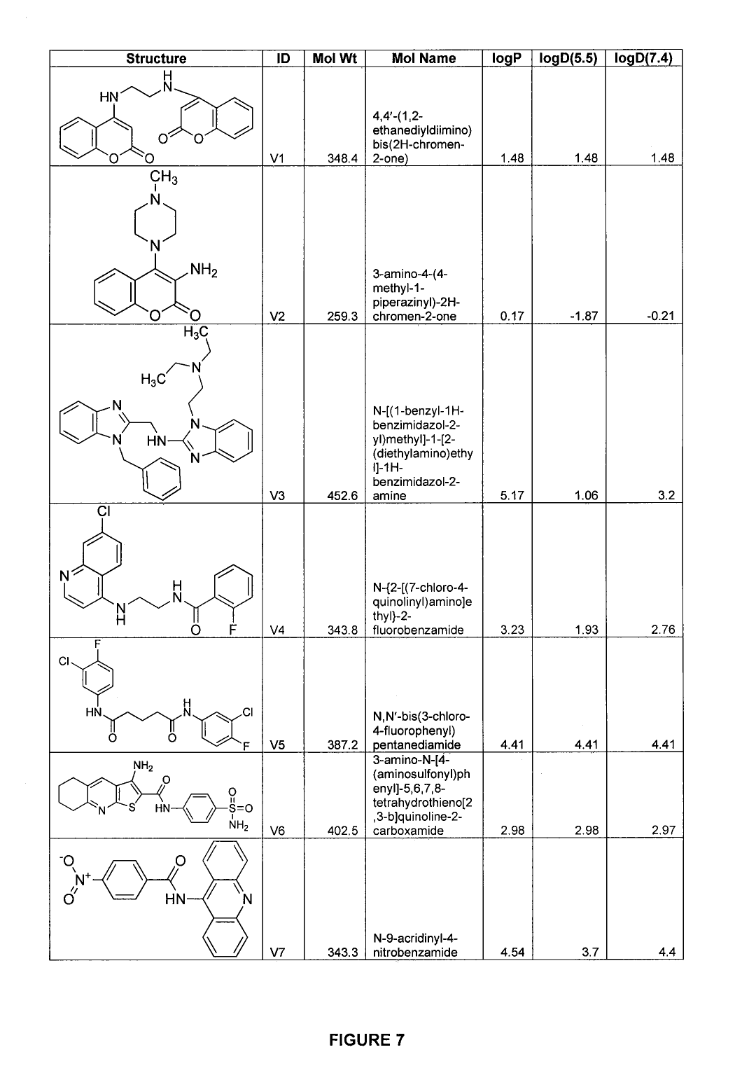

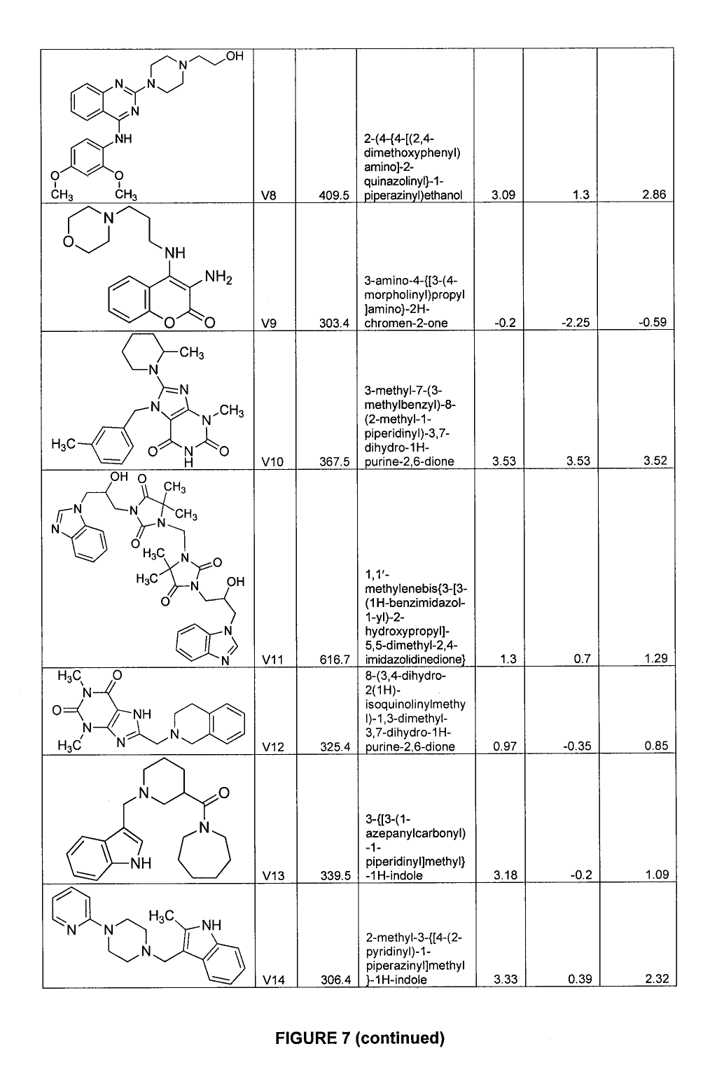

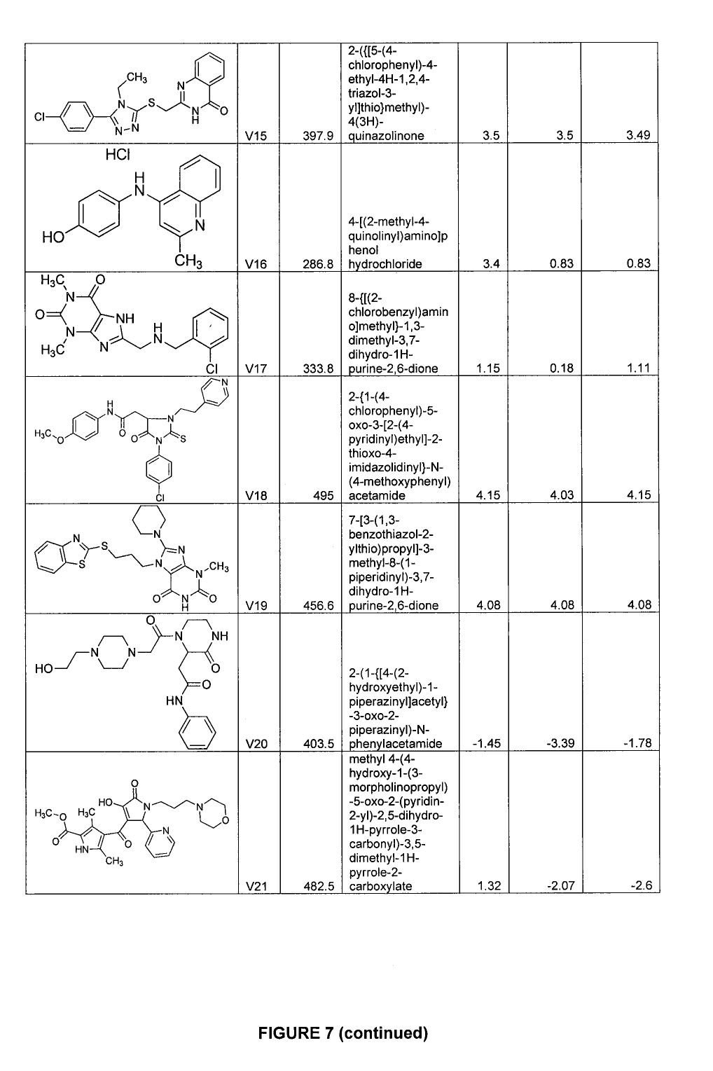

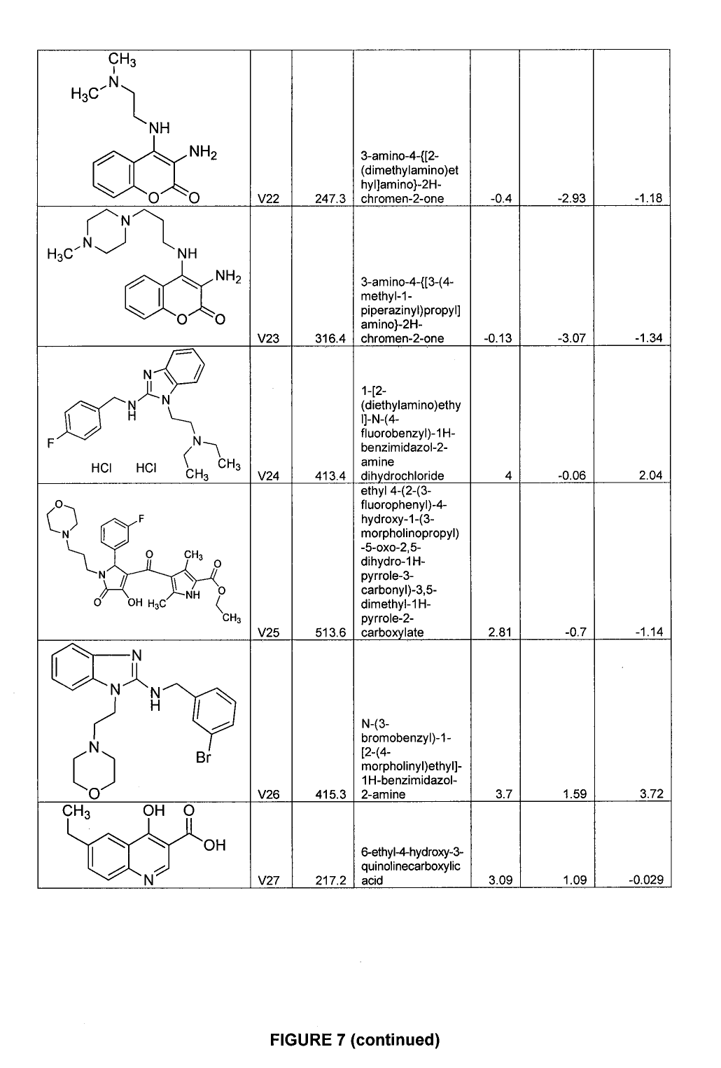

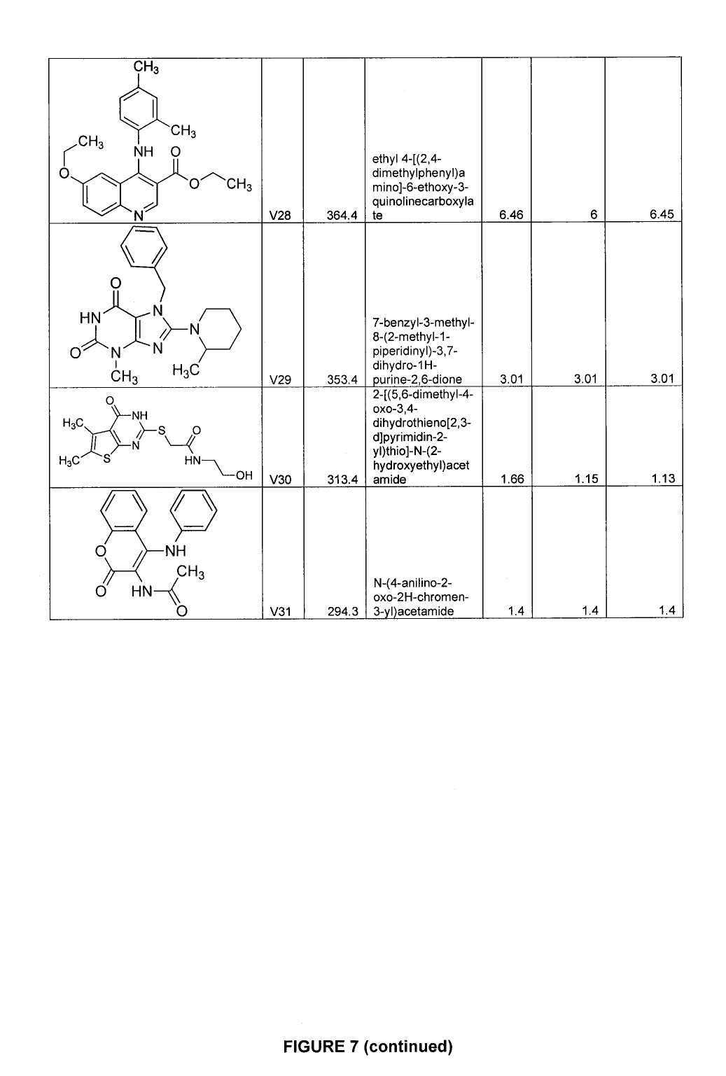

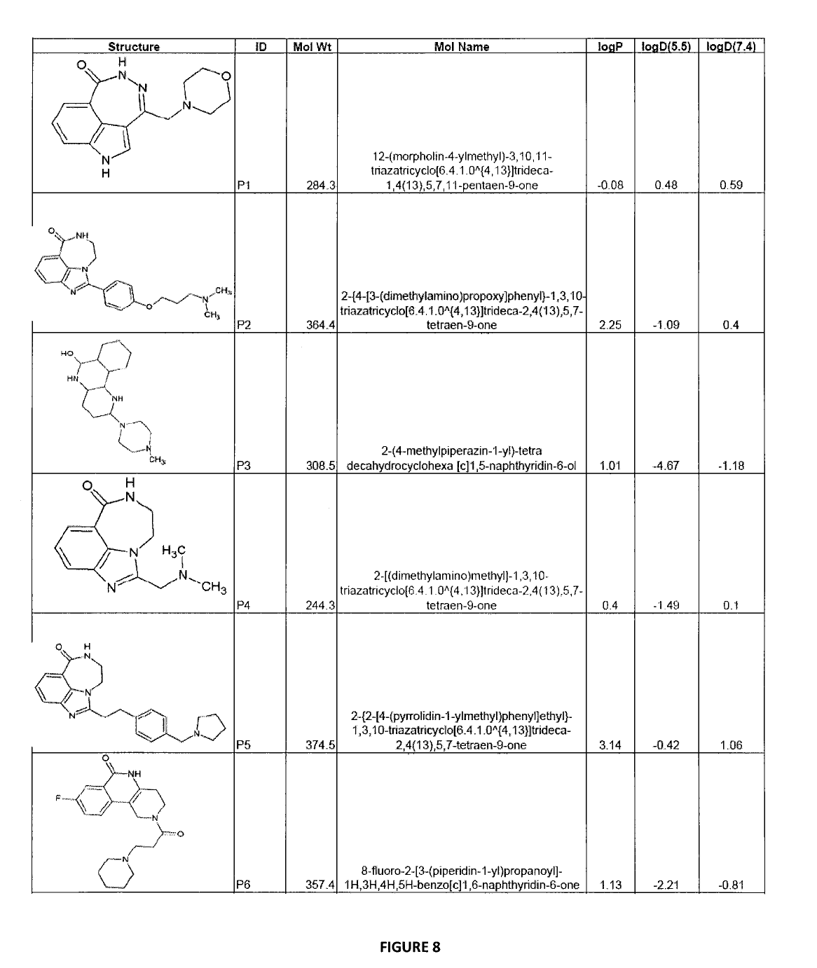

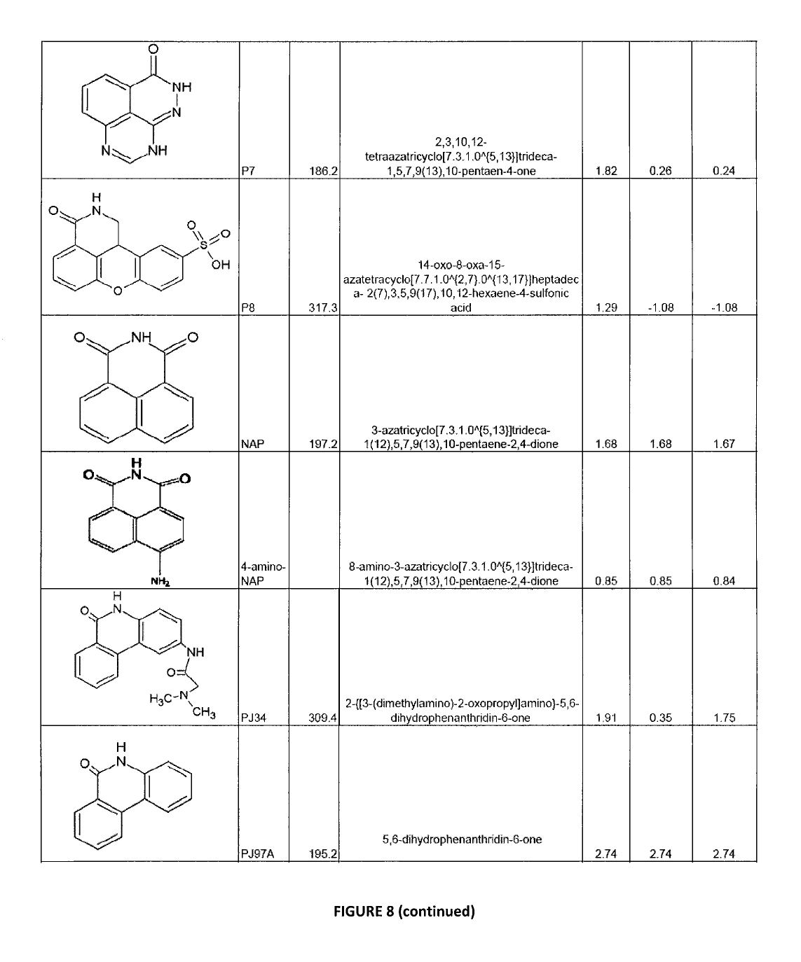

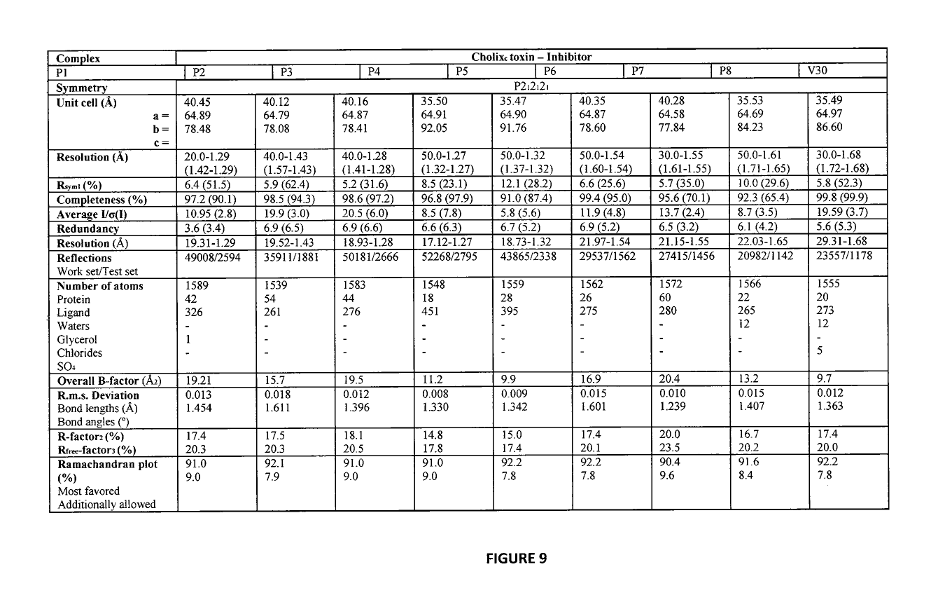

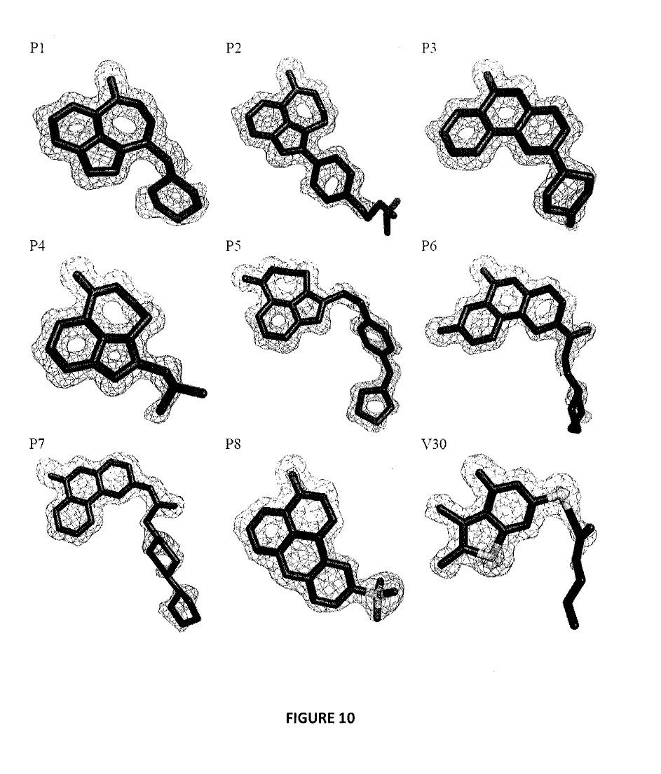

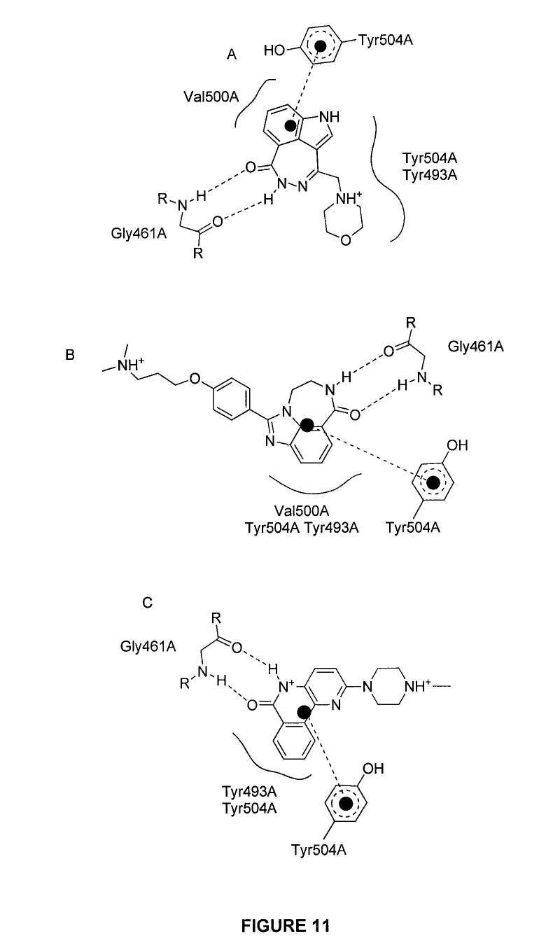

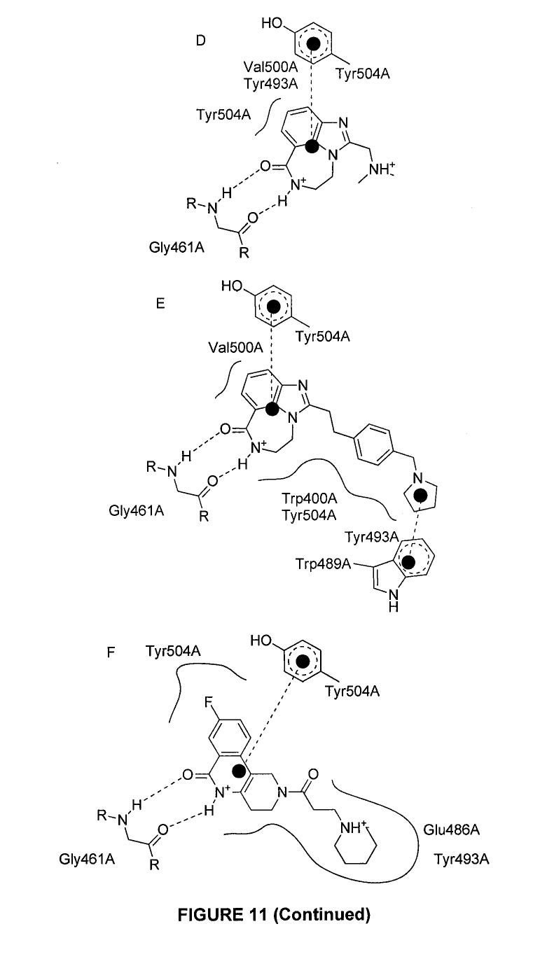

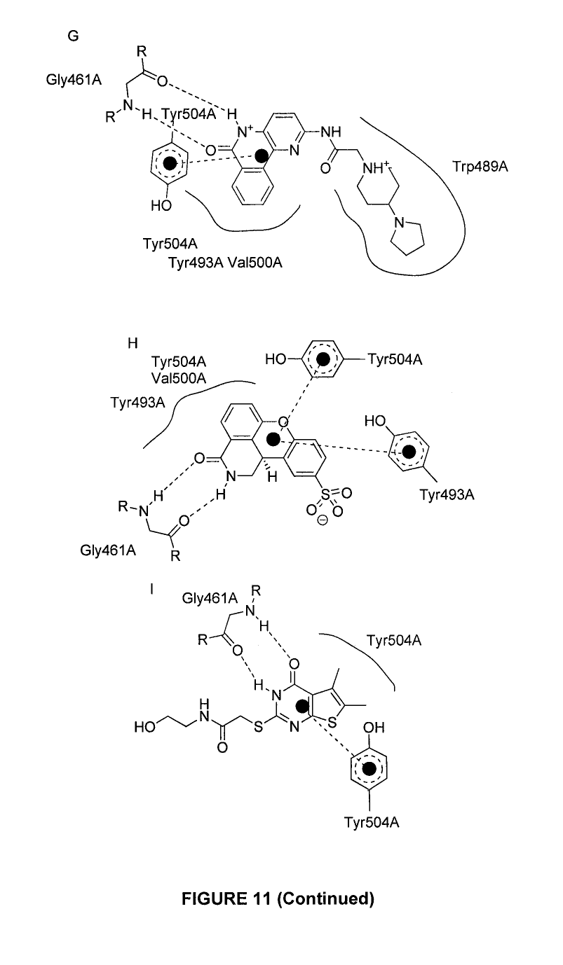

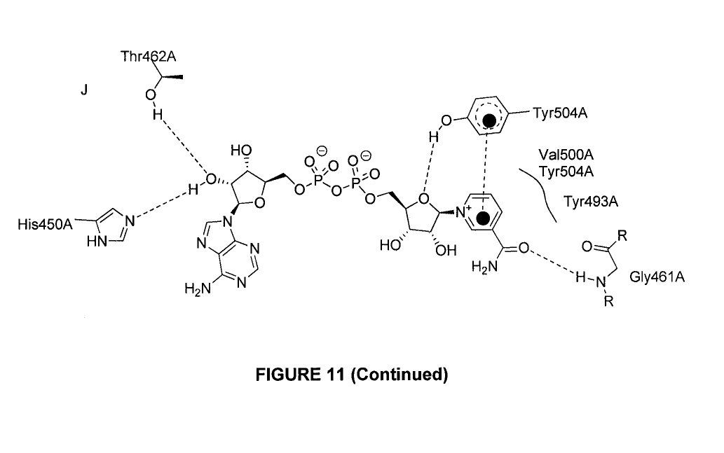

In accordance with this aspect of the application, two DT groups of mARTs targeting elongation factor 2 in the mammalian cell were considered in order to identify the most efficient antivirulence compounds to Exo A. ExoA, a well-characterized factor produced by P. aeruginosa, and cholix, a new mART toxin from V. cholerae recently identified in silico ¹³, which, along with DT, show nearly identical enzyme activity and inhibitor specificity ^{12,13,17-19} were used. Using the 1.25 Å co-crystal structure of cholix toxin with PJ34 inhibitor (PDB:2Q6M—a known poly-ADP-ribose polymerase (PARP) inhibitor) as a template, a virtual screen of over 500,000 commercial compounds identified 72 prospective inhibitors. After filtering for chemical stability and redundancy, 31 compounds were tested to assess their protective effect (Table 1, FIGS. 7, 8). Crystal structures of 9 novel inhibitors in complex with cholix toxin clearly demonstrate their binding within the toxin active site (FIGS. 10, 11). The high-resolution crystal structures of the best inhibitors in complex with cholix toxin thus revealed important criteria for inhibitor binding and mechanism of action.

Of course, in view of the above, a person skilled in art will understand that any toxin which is structurally and/or functionally similar to Exo A will also be inhibited, either completely or partially, by the antivirulence compounds identified in the present application.

Therefore, the antivirulence compounds of the present application which demonstrated a protective effect against ExoA in mammalian cells include compounds with any protective effect.

In its most general embodiment, the present application includes a method to inhibit bacterial virulence factors comprising administering an effective amount of an antivirulence compound to a subject or cell in need thereof. Another embodiment of the present application is a use of an antivirulence compound to inhibit bacterial virulence factors and a use of an antivirulence compound to prepare a medicament to inhibit bacterial virulence factors.

In an embodiment, an antivirulence compound of the present application, which demonstrated a protective effect against ExoA in mammalian cells, is a compound of the Formula I:

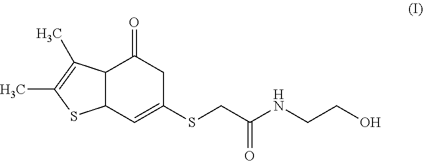

or a pharmaceutically acceptable solvate and/or prodrug thereof. Accordingly, in an embodiment of the application, there is included a method to inhibit bacterial virulence factors comprising administering an effective amount of a compound of Formula I, or a pharmaceutically acceptable solvate and/or prodrug thereof, to a subject or cell in need thereof. Another embodiment of the present application is a use of a compound of Formula I, or a pharmaceutically acceptable solvate and/or prodrug thereof, to inhibit bacterial virulence factors and a use of a compound of Formula I, or a pharmaceutically acceptable solvate and/or prodrug thereof, to prepare a medicament to inhibit bacterial virulence factors. In an embodiment of the application, the compound of Formula I is the compound V30 shown in FIG. 7, or a pharmaceutically acceptable solvate and/or prodrug thereof.

In an embodiment, an antivirulence compound of the present application, which demonstrated a protective effect against ExoA in mammalian cells, is a compound of the Formula II:

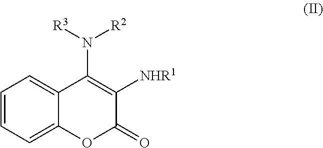

wherein:
R¹ is selected from H and C(O)C₁₋₄alkyl;
R² is H and R³ is selected from aryl and (CH₂)ₙNR⁴R⁵, or R² and R³, together with the nitrogen atom to which they are attached, form a piperidinyl, piperazinyl or morpholinyl ring;
R⁴ and R⁵ are independently selected from H, C₁₋₄alkyl or, together with the nitrogen atom to which they are attached, form a piperidinyl, piperazinyl or morpholinyl ring; and
n is 1, 2, 3 or 4,
or a pharmaceutically acceptable salt, solvate and/or prodrug thereof, to a subject or cell in need thereof.

Accordingly, in an embodiment of the application, there is included a method to inhibit bacterial virulence factors comprising administering an effective amount of a compound of Formula II, or a pharmaceutically acceptable solvate and/or prodrug thereof, to a subject or cell in need thereof. Another embodiment of the present application is a use of a compound of Formula II, or a pharmaceutically acceptable solvate and/or prodrug thereof, to inhibit bacterial virulence factors and a use of a compound of Formula II, or a pharmaceutically acceptable solvate and/or prodrug thereof, to prepare a medicament to inhibit bacterial virulence factors.

In an embodiment of the application, R¹ is selected from H, C(O)CH₃ and C(O)CH₂CH₃.

In another embodiment of the application, R² is H and R³ is selected from phenyl and (CH₂)ₙNR⁴R⁵, wherein R⁴ and R⁵ are independently selected from H, CH₃ and CH₂CH₃ or, together with the nitrogen atom to which they are attached, form a piperidinyl, piperazinyl or morpholinyl ring and n is 2 or 3.

In another embodiment of the application R² and R³, together with the nitrogen atom to which they are attached, form a piperidinyl, piperazinyl or morpholinyl ring.

In another embodiment of the application the compound of Formula II is selected from:

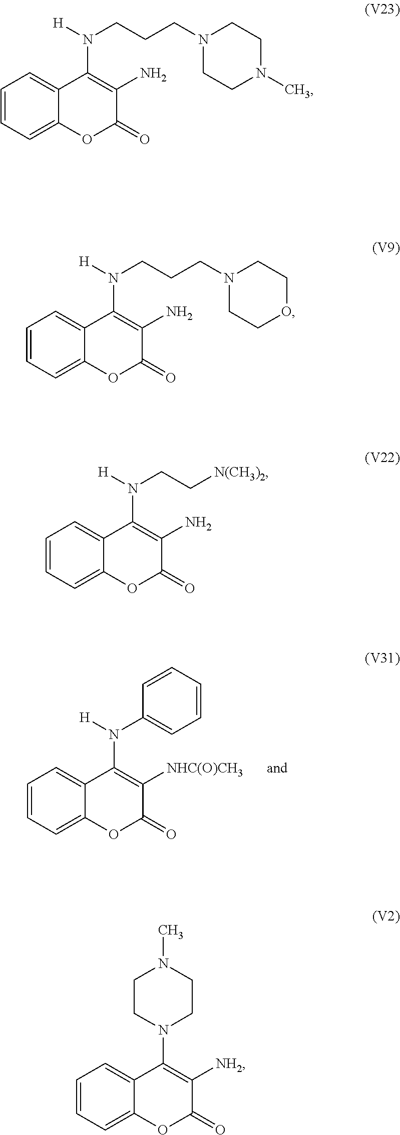

or a pharmaceutically acceptable salt, solvate and/or prodrug thereof.

In another embodiment of the application, the compound of Formula II is

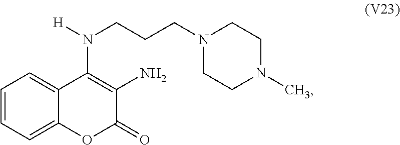

or a pharmaceutically acceptable salt, solvate and/or prodrug thereof.

In another embodiment of the application, the bacterial virulence factor is Exo A.

In another embodiment of the application, there is included a method of preventing and/or treating a disease state caused by a bacterial infection comprising administering an effective amount of one or more compounds selected from a compound of Formula I and a compound of Formula II, and pharmaceutically acceptable salts, solvates and/or prodrugs thereof to a subject or cell in need thereof. Another embodiment of the present application is a use of one or more compounds selected from a compound of Formula I, and a compound of Formula II, and pharmaceutically acceptable salts, solvates and/or prodrugs thereof, to inhibit bacterial virulence factors and a use of one or more or more compounds selected from a compound of Formula I and a compound of Formula II, and pharmaceutically acceptable salts, solvates and/or prodrugs thereof, to prepare a medicament to inhibit bacterial virulence factors. In an embodiment, the bacterial infection is, for example, a P. aeruginosa infection.

Yet another embodiment of the present application is a use of V30, V23, V9, V22, V31 or V2, or a pharmaceutically acceptable salt, solvate and/or prodrug thereof, to prevent and/or treat a disease state caused by a bacterial infection, for example a P. aeruginosa infection.

Yet another embodiment of the present application is to provide a method to treat or prevent a disease state caused by a bacterial infection comprising administering an effective amount of V30, or a pharmaceutically acceptable salt, solvate and/or prodrug thereof, to a subject or cell in need thereof.

Also included in the present application is a pharmaceutical composition for use to inhibit bacterial virulence factors comprising one or more compounds of Formula I or Formula II, or a pharmaceutically acceptable salt, solvate and/or prodrug thereof, in combination with a pharmaceutically acceptable carrier.

Also included in the present application is a pharmaceutical composition for use to prevent and/or treat a disease state caused by a bacterial infection comprising one or more compounds of Formula I or Formula II, or a pharmaceutically acceptable salt, solvate and/or prodrug thereof, in combination with a pharmaceutically acceptable carrier.

The term “pharmaceutically acceptable” means suitable for or compatible with the treatment of subjects, in particular mammals, including humans.

The term “pharmaceutically acceptable salt” means an acid or basic addition salt, which is suitable for or compatible with the treatment of subjects, in particular mammals, including humans.

The term “pharmaceutically acceptable acid addition salt” as used herein means any non-toxic organic or inorganic salt of any basic compound. Basic compounds that form an acid addition salt include, for example, compound comprising an amine group. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “pharmaceutically acceptable basic addition salt” as used herein means any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine, trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

In general, prodrugs will be functional derivatives of parent compounds which are readily convertible in vivo into the parent compound from which it is notionally derived. Prodrugs include, for example, conventional esters formed with an available, carboxylic acid, hydroxy and/or amino group. For example, available OH and/or NH₂ groups in a compounds is acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, prodrugs are those in which the carboxylic acid, hydroxy and/or amino groups in a compound is masked as a group which can be converted to carboxylic acid, hydroxy and/or amino groups in vivo. Conventional procedures for the selection and preparation of suitable prodrugs are described, for example, in “Design of Prodrugs” ed. H. Bundgaard, Elsevier, 1985.

As used herein, the phrase “effective amount” or “therapeutically effective amount” means an amount effective, at dosages and for periods of time necessary to achieve the desired result. For example in the context or treating a disease state caused by a bacterial infection, an effective amount is an amount that for example, treats, prevents or inhibits the disease state caused by the bacterial infection, compared to the response obtained without administration of the compound(s). Effective amounts may vary according to factors such as the disease state, age, sex, weight of the subject. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The term “treating” or “treatment” as used herein and as is well understood in the art, means an approach for obtaining beneficial or desired results, including clinical results. Beneficial or desired clinical results include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. Treatment methods comprise administering to a subject a therapeutically effective amount of one or more compounds or compositions described in the present application and optionally consist of a single administration, or alternatively comprises a series of applications. For example, the compounds or compositions described herein are administered at least once a week, from about one time per week to about once daily to about four times daily for a given treatment. The length of the treatment period depends on a variety of factors, such as the severity of the disease, the age of the patient, the concentration, the activity of the compound(s), and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required.

The term “subject” as used herein includes all members of the animal kingdom including mammals, and suitably refers to humans.

As used in this specification and the appended claims, the singular forms “a”, “an” and “the” include plural references unless the content clearly dictates otherwise. Thus for example, a composition containing “a compound” includes a mixture of two or more compounds.

In compositions comprising an “additional” or “second” component, the second component as used herein is chemically different from the other components or first component. A “third” component is different from the other, first, and second components, and further enumerated or “additional” components are similarly different.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including” and “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Further, terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

All references cited herein are incorporated by reference in their entirety.

### Example 1

### Characterization of Antivirulence Compounds Inhibiting P. aeruginosa Exotoxin (ExoA) and Cholix Toxin

1. Materials and Methods

### 1.1 Strains and Media

Saccharomyces cerevisiae W303 (MATa, his3, ade2, Ieu2, trp1, ura3, can1), ERG6-(MATa, his3, leu2, met15, ura3, erg6::KanMX), MTID:2955 (MATa, leu2, trp1, can1, ura3, ade2, his3, pdr1D::NAT, pdr3D::URA3), 2775 (MATa, his3, Ieu2, Iys2, ura3, MNN6::KanMX) and 7034 (MATa, his3, leu2, lys2, ura3, MNN4::KanMX) were grown on yeast-peptone-dextrose or synthetic dextrose (SD) drop-out medium. Human lung epithelial cells (C38) were cultured as previously described 20 in LHC-8 supplemented with 5% fetal bovine serum.

### 1.2 PARP Inhibitor Library

A small, directed poly-ADP-ribose polymerase library of 8 compounds was a gift from Guilford Pharmaceuticals (Baltimore, Md.). They were numbered P1-P8 to indicate that these compounds originated from this series.

1.3 P. aeruginosa Drug Sensitivity Assay

Overnight cultures of P. aeruginosa strain PA103 were streaked onto LB plates and grown overnight at 37° C. A single colony was selected from the plate and grown overnight in 5 ml of LB broth at 37° C. One hundred μL of the overnight culture was used to inoculate 250 mL of fresh LB broth. The inoculated media was added to sterile culture tubes in 5 mL aliquots. Inhibitors were added to each aliquot at a final concentration of 50 μM and incubated overnight at 37° C. Growth was deemed positive if the OD_{600 nm} was comparable (within 0.2 Abs units) to the untreated P. aeruginosa culture. DMSO (0.5%) was used as the negative control and tetracycline (5 μg/mL) and gentamycin (10 μg/mL) as positive controls.

### 1.4 Protein Purification of Cholix Toxin

Full-length and catalytic fragment (cholix_{c}) of cholix toxin were purified as previously described²¹. Non-recombinant, full-length exotoxin A was purchased from Sigma (Sigma Chemical, St. Louis, Mo.).

### 1.5 IC₅₀ Determination for Inhibitory Compounds

The concentration of inhibitor that reduced enzymatic activity by 50%, the IC₅₀ value, was measured as previously described¹⁷, with some modifications. The reaction mixtures (70 μL total volume) consisted of 300 μM ε-NAD, 10 μM eEF2 and a range of inhibitor concentrations in 20 mM Tris, 85 mM KCl, pH 7.9 buffer. The reaction mixtures were incubated at 25° C. for 5 min in disposable Ultra-micro UV cuvettes (Brand Scientific, Essex, Conn.) and the reactions were initiated by the addition of 5 nM of cholix toxin. The IC₅₀ value was determined by fitting the data to Boltzman Sigmoidal function by nonlinear regression with Origin 6.1 (OriginLab, Northampton, Mass.).

### 1.6 Calculated Kᵢ Values

Since the IC₅₀ value is not a direct indicator of affinity, these values were converted to Kᵢ values according to the Cheng-Prusoff equation²²: Kᵢ=IC₅₀/(1+[S]/K_{M}), where [S] is the NAD⁺ concentration and K_{M} is for the NAD⁺ substrate.

1.7 Calculated Log P and Log D values

The log P=log₁₀(partition coefficient), partition coefficient=[compound]_{octanol}/[compound]_{water} and was calculated (c log P) using an on-line c log P calculator by ChemAxon Ltd. The log D pH profiles were generated by the same software and represent the calculated log distribution coefficient, log D=log₁₀(distribution coefficient) where distribution coefficient, Σ[microspecies]_{octanol}/Σ[microspecies]_{water}. Calculated log D values (pH 5.5 and 7.4) were shown for the most active inhibitor compounds in Table 2 and for all tested compounds in FIGS. 7 and 8.

### 1.8 Virtual Screen

A virtual screen was conducted using GLIDE 5.0 (virtual docking program, Schrodinger, N.Y.) against the cholix_{c} structure (PDB code: 2Q6M). The approach was divided into the following steps: First, the Chembridge, Chemdiv, and Enamine libraries of 450,000, 750,000, and 850,000 compounds, respectively, were merged into a non-redundant data set of approximately 1,500,000 unique molecules. Molecular descriptors were calculated and compounds that did not satisfy the Lipinski rules, or have calculated Log S values lower than −6.5 (indicative of poor aqueous solubility) were filtered-out. The resulting library of about 500,000 drug-like molecules with favorable development potential constituted the virtual screening library. Second, each compound within the assembled library was docked to the active site of the enzyme using the GLIDE program, and assigned a score (a predicted pseudo-binding energy) that reflects the quality of the fit, and accounts for shape complementarity, Coulomb and continuum electrostatics, hydrogen bonding networks, and entropic penalty upon ligand binding.

### 1.9 Yeast-Based Compound Screen

Saccharomyces cerevisiae cells (strains W303, ERG6⁻, MTID:2955, 2775 and 7034) expressing the catalytic domain of P. aeruginosa ExoA (ExoA_{c}) were cultured in the presence of 50 μM of each compound and 1% dimethyl sulfoxide (DMSO) in 96-well plates for 48 hours as previously described^{19,23}.

### 1.10 c Log P and Log D Calculations

The calculated log P (c log P) values were obtained by using an on-line program at http://intro.bio.umb.edu/111-112/OLLM/111F98/newclogp.html. The log D pH profiles were generated by the same software and represent the calculated log distribution coefficient, log D=log₁₀(distribution coefficient) where distribution coefficient, D=Σ[microspecies]octanol/Σ[microspecies]water. Calculated log D values were shown for inhibitor compounds at pH 5.5 and 7.4.

### 1.11 Two-Dimensional Visualization

Two-dimensional cholix-inhibitor visualization was achieved by removing inhibitor coordinates from the respective pdb file, using Open Babel (http://openbabel.sourceforge.net/) to convert these coordinates to structure data format (*.sdf) and then drawing the complex using PoseView (http://poseview.zbh.uni-hamburg.de/). Note that water molecules, if any, are not shown.

### 1.12 Mammalian C38 Cell Drug Sensitivity Assay

The C38 cells were cultured in 25 cm² culture flasks containing LHC-8 media with 5% fetal bovine serum at 37° C. in 5% CO₂ until 90-100% confluency. The cells were washed with phosphate-buffered saline (PBS) and then detached from the culture flask by treating the cells with Puck's Saline Solution containing 0.02% EDTA. The cell suspension was diluted to a final density of 1.0×10⁵ cells/mL and 100-μL aliquots were used to seed a 96-well plate. The cells were then grown in the presence of 50 μM of inhibitor (nonpolar compounds required 0.5% DMSO for solubility). If the inhibitor compound reduced cell viability (using the MTT assay) to less than 70%, then the compound was considered toxic.

### 1.13 LD₅₀ for ExoA Against C38 Cells

The LD₅₀ is the lethal dose of toxin that kills 50% of the C38 cells. The C38 cells were cultured as described above except that full-length ExoA was added to the wells at 1-2000 ng/mL and the cells were allowed to grow for 96 h after which cell viability was determined using an MTT dye-based colorimetric assay¹⁹. The LD₅₀ values were determined by extrapolation from each corresponding line-of-best-fit (Sigmoidal function in Origin 6.1), and the average values reported±the standard deviation.

### 1.14 Inhibitor Cytoprotection in Mammalian Cells

C38 cells were cultured in the presence of varying concentrations of ExoA as previously described¹⁹ with the addition of each compound at 50 μM. Compounds V1-V31, NAP and PJ97A were assayed with 0.5% DMSO in the culture medium whereas compounds PJ34 and P1-P8 were assayed without DMSO. Cell viability was determined using an MTT dye-based colorimetric assay as previously described¹⁹.

### 1.15 Determination of Half-Maximal Effective Concentration (EC₅₀)

C38 cells were cultured in the presence of 650 ng/mL of ExoA (approximately 10-times the LD₅₀) in 96-well plates for 96 h. Each active inhibitor compound (NAP, V30, P1, P4, P5, P6) was added to the growth medium to give final concentrations varying from 0.5-100 μM. Cell viability was determined using the same method as the cytoprotection assay. The EC₅₀ values were extrapolated from each corresponding line-of-best-fit (Sigmoidal function in Origin 6.1), and the average values reported±the standard deviation.

### 1.16 Crystallization, Data Collection and Refinement of Cholix_{c}-Inhibitor Complexes

The purified cholix_{c} (6.6 mg/mL, final concn) was co-crystallized with the inhibitor compounds (15 mM, final concn) by the sitting drop vapour diffusion method against reservoirs containing 15-18% PEG-8000 and 10-20 mM KH₂PO₄ at 19° C. Before flash freezing in liquid N₂, the crystals were transferred to either paratone-N (Hampton Research) or (25% Glycerol, 16% PEG-10.000 and 0.2 M KH₂PO₄) for cryo-protection. All data sets were collected at beamline ID-08 (Canadian Light Source, Saskatoon, Canada) except for cholix_{c}-V30, which was collected at 1911-2 (MAX-lab, Lund, Sweden). The data were processed and reduced with XDS and XSCALE²⁴. All structures were solved by molecular replacement using either Molrep ²⁵ or Phaser ²⁶ with the cholix_{c} structure in complex with PJ34 (PDB entry 2Q6M) as a search model. Finally, the structure was iteratively rebuilt in Coot ²⁷ and refined using Refmac5 ²⁸. Structures with a resolution of 1.32 Å or higher were refined anisotropically whereas all other structures were refined isotropically including Translation/Libration/Screw (TLS) motion determination with the cholix_{c} structure divided into 10 groups.

2. Results and Discussion

### 2.1 Inhibitor Libraries.

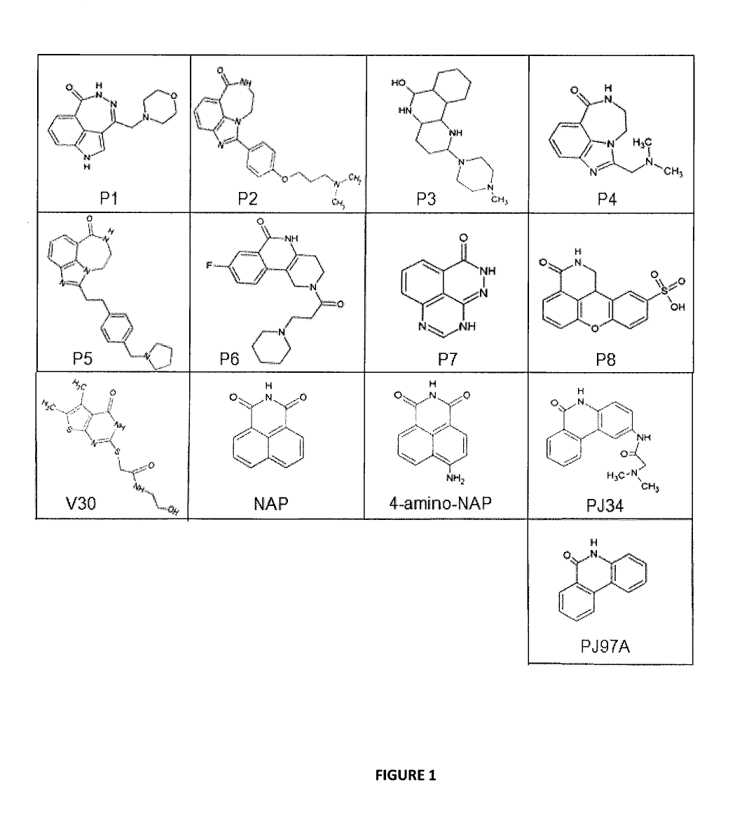

In this study, there were two libraries of compounds that were used. The first, designated the “P-series” was a small library of known PARP inhibitors (FIG. 1) and the second, designated the “V-series” was a library of commercially available compounds generated from a virtual screen conducted based on the crystal structure of cholix toxin with PJ34 inhibitor (PDB: 2Q6M). In addition, two well characterized mART inhibitors, 1-8-naphthalimide (NAP)¹⁹ and N-(6-oxo-5,6-dihydrophenanthridin-2-yl)-(N,N-dimethylamino)acetamide hydrochloride (PJ34)⁷ were included as controls for the in vitro and cell-based studies (FIG. 1). Also, a polar derivative of NAP, 4-amino-NAP, and the nonpolar parent of PJ34, PJ97A, were also tested (FIG. 1). It was previously demonstrated that the P-series compounds compete with the NAD⁺ substrate in ExoA/cholix by binding within the nicotinamide binding site within these enzymes^{17,18}. The virtual screen approach identified 72 hits as prospective inhibitors which were filtered based upon chemical stability and redundancy and 31 compounds were then tested experimentally (Table 1, FIG. 7).

All compounds were screened in yeast expressing the catalytic domain of ExoA ¹⁹ as well as against C38 cells dosed with the toxin. Compounds found to restore growth to 50-75% (++++), 25-50% (++) and 5-25% (+) were scored. Compounds showing less than 5% growth were scored (−). Compounds capable of protecting C38 cells from ExoA concentrations greater than 1000 ng/mL were scored (++++), 500-1000 (+++), 250-500 ng/ml (++) and 100-250 ng/ml (+). Compounds showing protection at less than 100 ng/mL toxin were scored (−). Protection was assessed by determining the ability of each inhibitor to increase the LD₅₀ of ExoA. Toxic indicates the inhibitor was cytotoxic to the cells and is defined as any compound that reduced cell viability to less than 30%. All compounds were screened at a final concentration of 50 μM. Yeast strains W303, ERG6⁻, MTDID:2955, 2775 and 7034 were screened in duplicate and no significant differences in protection were observed.

A small, in-house, poly-ADP-ribose polymerase (PARP) inhibitor library of 8 compounds was also tested and it was found that several of these PARP inhibitors showed potent mART inhibition both in vitro and in cell-based assays.

### 2.2 Inhibitor Activities in Yeast

All potential inhibitors (both the P- and V-series) were screened in five different yeast strains, previously developed to facilitate compound delivery to the yeast cytoplasm, expressing the ExoA_{c}. However, there were no significant differences in the effects of inhibitors in these yeast strains so only the results of screens in the w303 strain are shown (Table 1). It is known that intracellular expression of the ExoA_{c} gene in yeast causes a growth-defect phenotype which can be used as a cell-based measure of mART toxicity in this model system¹⁹. Effective inhibitors of mART (ExoA_{c}) activity can be screened in this yeast-based system where such agents abrogate the growth-defect phenotype²⁹. The following yeast strains employed for initial screening were w303 (wild-type), ERG6″ (which lacks the Δ24-sterol C-methyltransferase), MTID:2955 (which lacks two master regulators in the expression of pleitropic drug response elements), and 2775 and 7034 (deficient in mannosylphosphate transferase); however, no differences between the various yeast strains were observed and so w303 was used as the tester strain for this study²³. In yeast, generally compounds from both the P- and V-series that were able to at least partially restore yeast growth (although V23 and V30 are exceptions) were nonpolar, lacked solubility in aqueous buffer, and were not ionizable (log D values; FIGS. 7 and 8) suggesting the cell wall acts as a physical barrier, particularly to polar molecules. This was most obvious in the structure/activity relationships of NAP and its more polar derivative, 4-amino-NAP, and between PJ97A and the more polar PJ34, where the nonpolar parent compound was active against ExoA_{c} in yeast, but the more polar derivative was considerably less active (Table 1, FIGS. 1, 7 and 8)^{30,31}. None of the polar P-series compounds (P1-P8) showed inhibitor efficacy in yeast against ExoA_{c} and in the V-series only compound V23 showed modest protection in yeast with weak protection afforded by compounds V15, V29, and V31. Yeast grow on less complex media, are genetically simpler, lacking many redundant systems of mammalian cells, and provide more rapid feedback as a screening tool; however, yeast are limited for identifying polar/ionizable inhibitors because their cell wall presents a barrier to the permeation of these compounds^{30,31}.

### 2.3 ExoA Cytotoxicity in Mammalian Cells and Inhibitor Protection

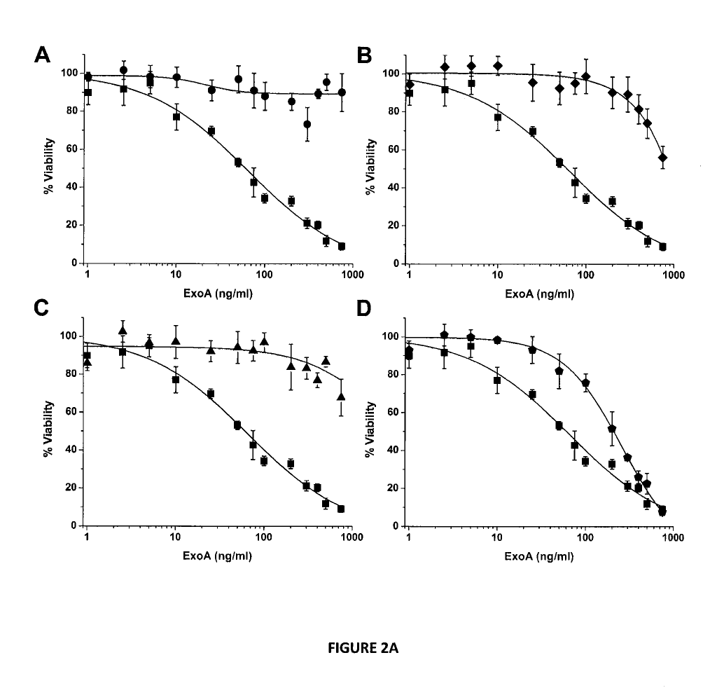

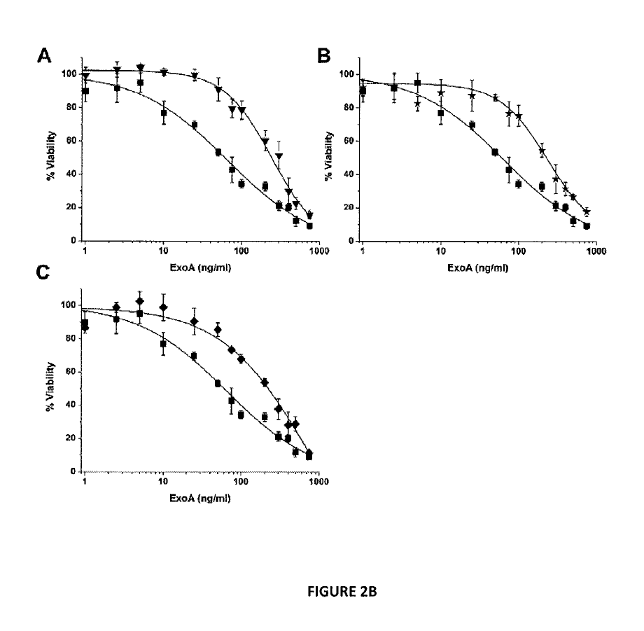

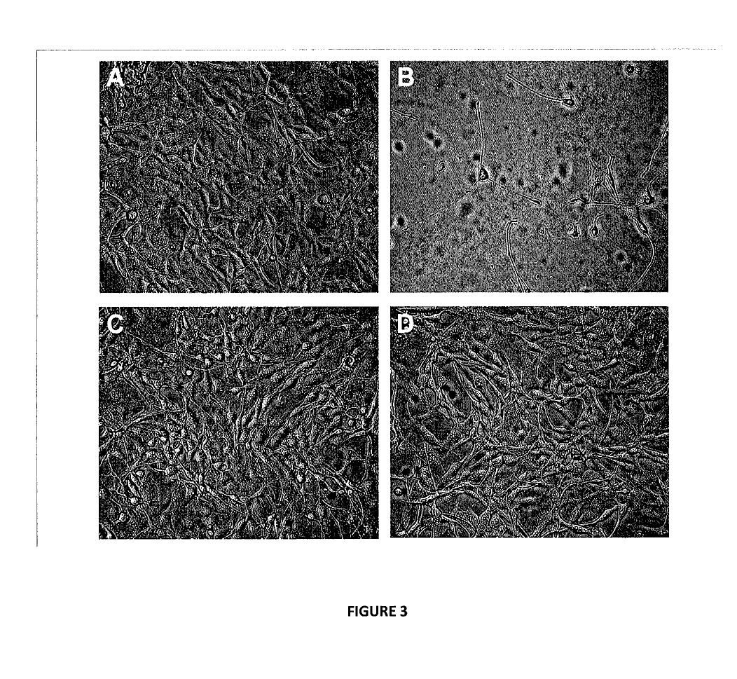

ExoA invades target mammalian cells through the recognition of the low-density lipoprotein receptor (LRP) in the plasma membrane³² and it kills the host cell by arresting protein synthesis and inducing apoptosis³³. ExoA showed an LD₅₀ of 65±12 ng/mL for the C38 cells (FIG. 2A A-C, control curve for ExoA with no inhibitor). NAP and V30 provided excellent protection of C38 cells against ExoA toxicity, even at very high toxin doses (FIG. 1; FIG. 2A A, C). At high doses (500 ng/mL), ExoA was highly toxic to the C38 cells (FIG. 3B) compared with the healthy control cells (FIG. 3A). Remarkably, compounds V30 and P1 completely protected the C38 cells from the lethal action of high doses of ExoA (FIG. 3 C, D).

### 2.4 V-Series Inhibitor Activities in Mammalian Cells

In C38 cells, virtual screen compound V30 showed excellent protection against ExoA with moderate activity exhibited by V9, V12, V15, V17, V23, and V24 (Table 1). Weak protection was shown by V10, V11, V16, V21, V22, V25, V26, V29, and V31 (Table 1). Therefore, V30, which was the only V-series inhibitor to show excellent protection of C38 from toxin, was chosen for further testing and quantification for in vitro inhibitory (IC₅₀) activity. NAP, a previously characterized, competitive inhibitor of ExoA,¹⁸ served as a known toxin inhibitor control; both V30 and NAP were remarkably similar in being able to protect C38 cells and increased the LD₅₀ values for ExoA by 30-40-fold (FIG. 2A A,C). Compound PJ97A, a derivative of the well-characterized ExoA/cholix inhibitor, PJ34¹⁷, also showed strong protection against ExoA but not as much as NAP and V30 (LD₅₀ increased nearly 12-fold for PJ97A) (Table 2, FIG. 2B). Compound V23 is an example of a modest ExoA inhibitor in C38 cells, and it was, in fact, the weakest in this active inhibitor group (FIG. 2D). Modest V-series inhibitors such as V12 increased the toxin LD₅₀ by about only 3-fold (Table 1; FIG. 2B).

### 2.5 P-Series Inhibitor Activities in Mammalian Cells

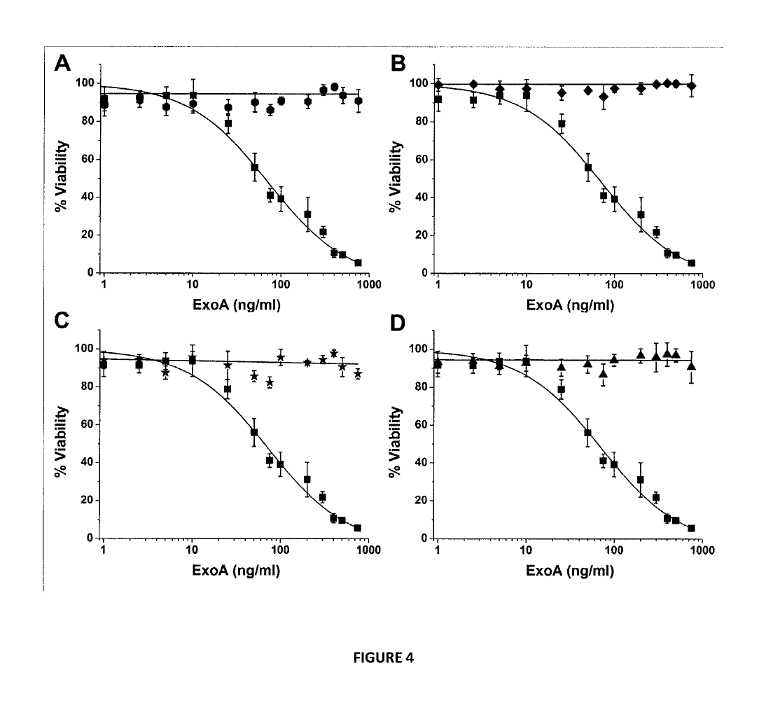

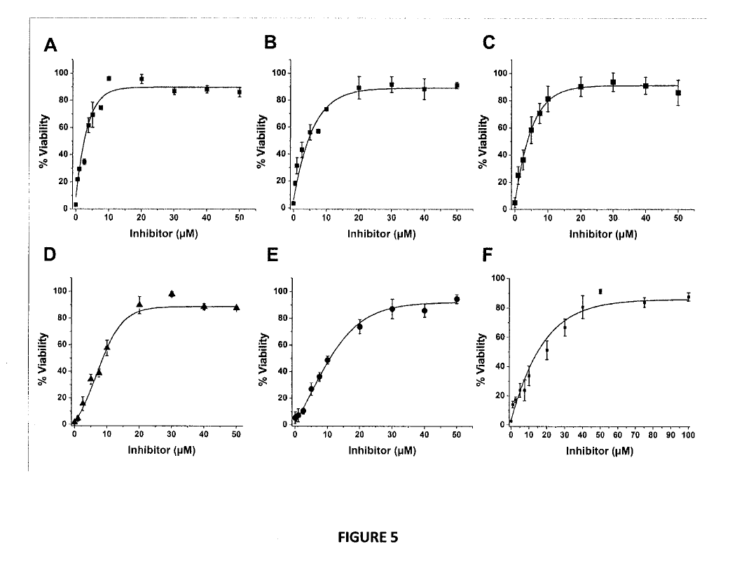

In the P-series library (FIG. 1), compounds P1, P4, P5 and P6 showed excellent protection (over 90%) at ExoA doses near 1000 ng/mL (ExoA LD₅₀=65 ng/mL) (FIG. 4A-D), which was even greater than the protection provided by both NAP and V30 inhibitors. Notably, compounds P2, P3, and P7 showed good IC₅₀ values against purified cholix (Table 2), but were toxic to C38 cells (Table 1, listed as toxic) and thus were excluded from cell-based testing. The C38-toxic compounds reduced viability to less than 10% and the nontoxic inhibitors did not reduce viability below 95% at the 50 μM dose (Table 1). Compound P8 also showed a good in vitro IC₅₀ value, but exhibited only a modest ability to protect C38 cells from ExoA (FIG. 1; Tables 1, 2).

### 2.6 Correlation of Cell-Based and In Vitro Inhibitor Characteristics

Several compounds from both V- and P-series showed good inhibitor efficacy with EC₅₀ values that ranged from 2.9-16.7 μM, including P1, P6, NAP, V30, P4, and P5 (FIG. 4A-F; Table 2). IC₅₀ values and binding affinities (K_{d}) were measured for these compounds in vitro for comparison with the cell-based results. NAP, P1 and

P6 compounds showed low-to-mid nM IC₅₀ values whereas P4, V30, and P5, gave higher values (960 nM, 2815 nM, and 4460 nM, respectively) (Table 2). The higher IC₅₀ values for these latter compounds were reflected in the corresponding Kᵢ values (inhibitor binding constants) and represent weaker binding to the toxin active site. However, this weaker affinity was not a factor in the ability of these compounds to block toxin activity in C38 cells (Tables 1, 2) at the 50 μM experimental dose. Interestingly, the inhibitor efficacy (EC₅₀ values) did not correlate with the inhibition constant (Kᵢ) of these compounds with the toxin target enzyme, indicating that the minimum requirement for a good inhibitor is competitive binding to the active site at 1 μM affinity or better (Table 2, Kᵢ values). Furthermore, EC₅₀ values reflect the pharmacokinetic properties of these compounds that involve a number of processes within the target cell such as the ability to cross a membrane, ability to diffuse to the target protein within the cell millieu and stability within the cell. It is notable that the K_{d} values of cholix with inhibitors also did not correlate with their corresponding Kᵢ values; this is likely because the Kᵢ reflects the binding constant of the inhibitor in complex with its protein target, eEF2 (taken from kinetic inhibition data), whereas the K_{d} is the binding constant for the free enzyme with inhibitor. Two compounds, P1 and P8, showed dual binding constants, indicating two distinct binding sites with approximately a 50-fold difference in affinity for cholix. One explanation for this phenomenon is that the high affinity site is the active site and the lower affinity site involves a region of the enzyme that is not the active site, but is located elsewhere on the protein surface. This has been observed for a number of enzymes during crystallization with substrate analogues/inhibitors³⁴.

The resulting library of mART inhibitors (Table 1) includes eight compounds that showed nearly 100% protection of mammalian cells against high doses of bacterial toxin (++++ or +++), six compounds that showed moderate protection (++), and 11 compounds that showed weak protection (+). Two compounds from the combined libraries showed good protection of yeast, two showed moderate, and three showed only weak protection from bacterial toxin (Table 1). Importantly, none of the inhibitors tested from both the virtual screen and the PARP library were toxic to P. aeruginosa at the doses administered to yeast and mammalian cells (see P. aeruginosa drug sensitivity assay in Materials and Methods). Although the mART toxin family shares a common NAD⁺ binding site with many mammalian enzymes including dehydrogenases, it is not likely that competitive inhibitors specific for the NAD⁺ binding site within these toxins will cause mammalian cell toxicity. This is because the toxin NAD⁺ binding site coordinates NAD⁺ in an unique, twisted horseshoe configuration ³⁵ that is very different from the extended NAD⁺ conformation associated with dehydrogenases, for instance.

### 2.7 Physico-Chemical Properties of Potential Toxin Inhibitors

The log partition coefficient (log P) is a measure of the differential solubility of a compound in immiscible solvents such as octanol and water and in drug discovery it is a useful parameter to understand the behaviour of drug molecules in cell culture and in the human body. According to Lipinski's rule of 5, the log P (c log P, calculated log P) for a drug candidate should be <5 and can be applied to neutral compounds³⁶; however, log D pH profiles should be used to estimate the bioavailability of ionizable compounds. The best inhibitors in C38 cells (Tables 1, 2) are ionizable compounds that were ambivalent towards aqueous or organic solvent (log D=−1.49-1.68; Tables 2, FIGS. 7 and 8); however, NAP and PJ97A are neutral compounds—the latter compound suffered from lower aqueous solubility (c log P_{PJ97A}=2.74; Tables 2, FIG. 8), likely compromising its bioavailability compared with NAP (c log P_{NAP}=1.68; Table 2, FIG. 8).

2.8 Structures of Inhibitors with Cholix Toxin

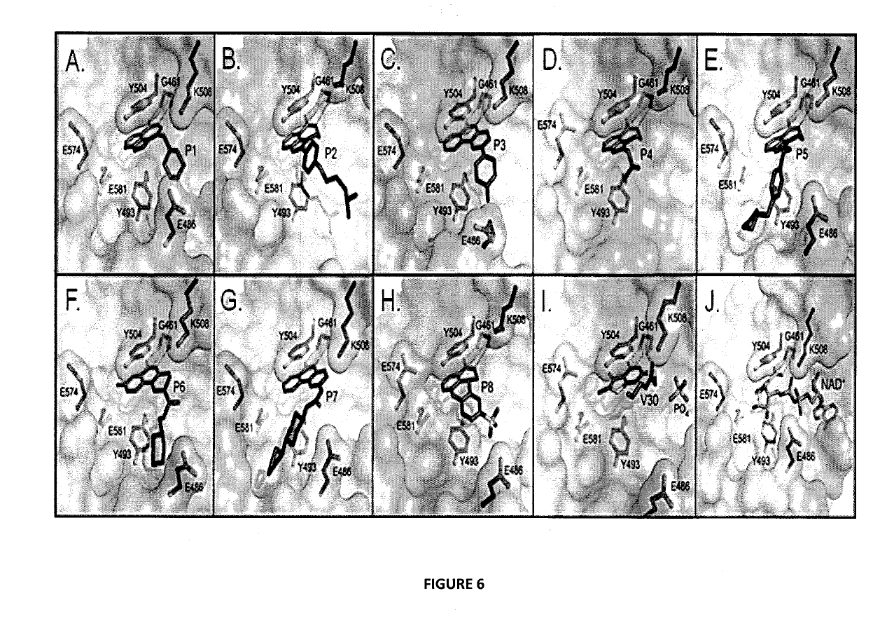

ExoA and cholix are closely related mART toxins that possess nearly identical biological activity¹³. However, cholix, produces superior crystal structures and was used as the model for structure determination of inhibitor complexes ¹⁹. The high-resolution crystal structures (1.27-1.68 Å) of cholix_{c} with inhibitors P1-P8, and V30 were solved by molecular replacement and provided strong support to the in vitro and cell-based inhibition data (Tables 1, 2, FIG. 10). These structures unequivocally demonstrated that these compounds are competitive inhibitors of NAD⁺ and bind within the nicotinamide-binding domain of cholix (FIG. 6 Al compared with FIG. 6J). The co-crystal structures of cholix_{c} with NAP and PJ34 were previously solved ^{13,19} and serve as useful references for understanding the binding of inhibitors P1-P8 and V30 within the cholix_{c} active site (FIG. 1, FIG. 6). The key features for these active-site inhibitors of ExoA include a benzamido group fused into a hetero-ring structure, which acts to mimic the nicotinamide moiety of NAD⁺¹⁷ by intercalating deeply within the nonpolar pocket while forming aromatic ring stacking interactions with the two conserved Tyr residues of cholix_{c} (Tyr 493 and Tyr 504; FIG. 1; FIG. 6J). These two Tyr residues flank the core of the inhibitor structures and provide a hydrophobic cradle for nonpolar, planar aromatic ligands such as NAP and compounds P1-P8 and V30 (FIGS. 1, 6, 10). Furthermore, in all compounds, a key interaction includes H-bonding between the inhibitor imide and the backbone groups of a conserved Gly residue (Gly461) within the Y—H-G motif of the active site core as seen for the NAD⁺ substrate (FIG. 6J) ¹⁹. Two dimensional drawings of the interactions of each inhibitor with the cholix active-site residues can be seen in FIG. 11, which help to illustrate the common mode of interaction of these inhibitory compounds within the toxin active site and help to explain their competitive inhibition kinetics (Table 2).

3. Conclusion

A known yeast-based approach for the identification and characterization of mART toxins from pathogenic bacteria was used as a screening method which provides great promise for the discovery and characterization of novel therapeutics against these toxins¹⁹. The utility of a virtual chemical screen in combination with the screening method detailed in the description of the present application identified lead compounds against mART toxins that show efficacy in cell-based systems. One of these compounds, V30, was identified as the best candidate for an antivirulence compound against the mART family of bacterial toxins. A small, directed PARP library of inhibitor compounds against ExoA was also tested. All of the compounds bound to the cholix active site and provided some protection in C38 cells (except for toxic compounds P2, P3, and P7), whereas only nonpolar compounds were effective in yeast cells (Table 1). From the data provided above, it was demonstrated that the ExoA/cholix inhibitors dock within the nicotinamide-binding pocket of the active site of these toxins and inhibit the mART enzymatic activity of purified toxin. Furthermore, the most efficacious compounds function as DT-group mART toxin inhibitors with prophylactic properties for human lung epithelial cells. The best compounds are not toxic to either the lung cells or the producing bacterial pathogen, in this case, P. aeruginosa, and hence have great potential as antivirulence agents for treating many bacterial infections and disease states. These newly discovered antivirulence compounds can now be exploited as new antivirulence therapeutics for treating bacterial disease states and infections ^{15,16}. To this effect, the efficacy of the antivirulence compounds can be further assessed in animal challenge studies. More specifically, the efficacy of the compounds to inhibit Exo A can be further assessed in mice using assays known in the art.

Although a preferred embodiment of the present application has been described in detail herein and illustrated in the accompanying drawings, it is to be understood that the application is not limited to this embodiment and that various changes and modifications could be made without departing from the scope and spirit of the present application.

TABLE 1
Overview of the cell-based results for potential ExoA inhibitors.

| | Protection in | Protection in | |
|---|---|---|---|
| Compound | C38 cells | Yeast | Source |
| NAP | ++++ | ++++ | ¹⁹ |
| 4-NAP | +++ | ++ | ¹⁹ |
| PJ97A | +++ | +++ | ³⁷ |
| PJ34 | − | − | ³⁷ |
| V1 | − | − | This study |
| V2 | + | − | This study |
| V3 | toxic | − | This study |
| V4 | toxic | − | This study |
| V5 | toxic | toxic | This study |
| V6 | − | − | This study |
| V7 | toxic | toxic | This study |
| V8 | − | − | This study |
| V9 | ++ | − | This study |
| V10 | + | − | This study |
| V11 | + | − | This study |
| V12 | ++ | − | This study |
| V13 | − | − | This study |
| V14 | − | − | This study |
| V15 | ++ | + | This study |
| V16 | + | − | This study |
| V17 | ++ | − | This study |
| V18 | − | − | This study |
| V19 | toxic | − | This study |
| V20 | − | − | This study |
| V21 | + | − | This study |
| V22 | + | − | This study |
| V23 | ++ | ++ | This study |
| V24 | ++ | − | This study |
| V25 | + | − | This study |
| V26 | + | − | This study |
| V27 | − | − | This study |
| V28 | toxic | toxic | This study |
| V29 | + | + | This study |
| V30 | ++++ | − | This study |
| V31 | + | + | This study |
| P1 | ++++ | − | This study |
| P2 | toxic | − | This study |
| P3 | toxic | − | This study |
| P4 | ++++ | − | This study |
| P5 | ++++ | − | This study |
| P6 | ++++ | − | This study |
| P7 | toxic | − | This study |
| P8 | + | − | This study |

TABLE 2
Comparison of K_{d}, Tₘ, IC₅₀, and EC₅₀ values for inhibitors of P. aeruginosa ExoA.

| | | | | ^{d}EC₅₀ | | ^{f}logD | ^{f}logD |
|---|---|---|---|---|---|---|---|
| Compd | ^{a}K_{d} (nM) | ^{b}Kᵢ (nM) | ^{c}IC₅₀ (nM) | (μM) | ^{f}clogP | pH_{5.5} | pH_{7.4} |
| P1 | 10 ± 2; 650 ± 50 | 22 ± 4 | 170 ± 30 | 2.9 ± 0.8 | −0.08 | 0.48 | 1.59 |
| P2 | 260 ± 10 | 63 ± 5 | 480 ± 40 | ^{e}n.d. | 2.25 | −1.09 | 0.4 |
| P3 | 1470 ± 30 | 90 ± 17 | 690 ± 130 | ^{e}n.d. | 1.01 | −4.67 | −1.18 |
| P4 | 1380 ± 30 | 132 ± 7 | 960 ± 50 | 12.6 ± 3.3 | 0.4 | −1.49 | 0.1 |
| P5 | 750 ± 10 | 582 ± 124 | 4460 ± 950 | 16.7 ± 1.9 | 3.14 | −0.42 | 1.06 |
| P6 | 1100 ± 20 | 80 ± 18 | 610 ± 140 | 3.4 ± 1.6 | 1.13 | −2.21 | −0.81 |
| P7 | 680 ± 40 | 118 ± 7 | 908 ± 118 | ^{e}n.d. | 1.82 | 0.26 | 1.24 |
| P8 | 160 ± 30; 5210 ± 1780 | 136 ± 3 | 1040 ± 136 | ^{e}n.d. | 1.29 | −1.08 | −1.08 |
| V30 | 931 ± 74 | 367 ± 3 | 2815 ± 22 | 8.8 ± 0.5 | 1.66 | 1.15 | 1.13 |
| NAP | 950 ± 30 | 12 ± 1 | 90 ± 10 | 3.8 ± 0.9 | 1.68 | 1.68 | 1.67 |
| PJ34 | 820 ± 54 | 37 ± 9 | 280 ± 70 | ^{e}n.d. | 1.91 | 0.35 | 1.75 |
| PJ97A | 393 ± 64 | 610 ± 175 | 4674 ± 175 | ^{e}n.d. | 2.74 | 2.74 | 2.74 |

^{a}The binding affinity of inhibitors to WT ExoA_{c} was measured by the quenching of the intrinsic Trp fluorescence caused by the binding of the ligand to the enzyme active site.
^{b}The absolute inhibition constant (Kᵢ) was calculated from the experimentally determined IC₅₀ values according to the following relationship, Kᵢ = IC₅₀/(1 + ([S_{NAD}]/K_{M(NAD)})²²; see Materials and Methods for details. The [NAD⁺] was 300 μM and the K_{M (NAD)} was 45 μM.
^{c}The IC₅₀ values were determined by fitting each dose-response curve to a Boltzmann Sigmoidal function in Origin 6.1.
^{d}The EC₅₀ values were determined for inhibitors added to C38 cells in the presence of 650 ng/ml of ExoA in 96-well plates for 96 h (see Materials and Methods for details).
^{e}n.d., not determined.
^{f}The logP and logD values were calculated as described in Materials and Methods.

### FULL CITATIONS FOR DOCUMENTS REFERRED TO IN THE APPLICATION

1. Raghavan, S., P. Manzanillo, K. Chan, C. Dovey, and J. S. Cox. 2008. Secreted transcription factor controls Mycobacterium tuberculosis virulence. Nature 454:717-721.
2. Tsigrelis, C., K. V. Singh, T. D. Coutinho, B. E. Murray, and L. M. Baddour. 2007. Vancomycin-resistant Enterococcus faecalis endocarditis: linezolid failure and strain characterization of virulence factors. J. Clin.Microbiol. 45:631-635.
3. Yahr, T. L. and M. C. Wolfgang. 2006. Transcriptional regulation of the Pseudomonas aeruginosa type III secretion system. Mol. Microbiol. 62:631-640.
4. Higgins, D. A., M. E. Pomianek, C. M. Kraml, R. K. Taylor, M. F. Semmelhack, and B. L. Bassler. 2007. The major Vibrio cholerae autoinducer and its role in virulence factor production. Nature 450:883-886.
5. Kollef, M. H. 2008. Broad-spectrum antimicrobials and the treatment of serious bacterial infections: getting it right up front. Clin. Infect. Dis. 47 Suppl 1:S3-13.
6. Benghezal, M., E. Adam, A. Lucas, C. Burn, M. G. Orchard, C. Deuschel, E. Valentino, S. Braillard, J. P. Paccaud, and P. Cosson. 2007. Inhibitors of bacterial virulence identified in a surrogate host model. Cell Microbiol. 9:1336-1342.
7. Maresso, A. W. and O, Schneewind. 2008. Sortase as a target of anti-infective therapy. Pharmacol. Rev. 60:128-141.
8. Cegelski, L., G. R. Marshall, G. R. Eldridge, and S. J. Hultgren. 2008. The biology and future prospects of antivirulence therapies. Nat. Rev. Microbiol. 6:17-27.
9. Escaich, S. 2008. Antivirulence as a new antibacterial approach for chemotherapy. Curr. Opin. Chem. Biol. 12:400-408.
10. Rappuoli, R. 2007. Bridging the knowledge gaps in vaccine design. Nat. Biotechnol. 25:1361-1366.
11. Clatworthy, A. E., E. Pierson, and D. T. Hung. 2007. Targeting virulence: a new paradigm for antimicrobial therapy. Nat. Chem. Biol. 3:541-548.
12. Yates, S. P., R. Jorgensen, G. R. Andersen, and A. R. Merrill. 2006. Stealth and mimicry by deadly bacterial toxins. Trends Biochem. Sci. 31:123-133.
13. Jorgensen, R., A. E. Purdy, R. J. Fieldhouse, M. S. Kimber, D. H. Bartlett, and A. R. Merrill. 2008. Cholix Toxin, a Novel ADP-ribosylating Factor from Vibrio cholerae. J. Biol. Chem. 283:10671-10678.
14. Fieldhouse, R. J. and A. R. Merrill. 2008. Needle in the haystack: structure-based toxin discovery. Trends Biochem. Sci. 33:546-556.
15. Steno, L. and A. Emili. 2008. Proteomic methods for drug target discovery. Curr. Opin. Chem. Biol. 12:46-54.
16. Diep, B. A. and M. Otto. 2008. The role of virulence determinants in community-associated MRSA pathogenesis. Trends Microbiol. 16:361-369.
17. Yates, S. P., P. L. Taylor, R. Jorgensen, D. Ferraris, J. Zhang, G. R. Andersen, and A. R. Merrill. 2005. Structure-function analysis of water-soluble inhibitors of the catalytic domain of exotoxin A from Pseudomonas aeruginosa. Biochem. J 385:667-675.
18. Armstrong, S., J. H. Li, J. Zhang, and A. R. Merrill. 2002. Characterization of competitive inhibitors for the transferase activity of Pseudomonas aeruginosa exotoxin A. J. Enzyme Inhib. Med. Chem. 17:235-246.
19. Turgeon, Z., D. White, R. Jorgensen, D. Visschedyk, R. J. Fieldhouse, D. Mangroo, and A. R. Merrill. 2009. Yeast as a tool for characterizing mono-ADP-ribosyltransferase toxins. FEMS Microbiol. Lett. 300:97-106.
20. Zeitlin, P. L., L. Lu, J. Rhim, G. Cutting, G. Stetten, K. A. Kieffer, R. Craig, and W. B. Guggino. 1991. A cystic fibrosis bronchial epithelial cell line: immortalization by adeno-12-SV40 infection. Am. J. Respir. Cell Mol. Biol. 4:313-319.
21. Jorgensen, R., Y. Wang, D. Visschedyk, and A. R. Merrill. 2008. The nature and character of the transition state for the ADP-ribosyltransferase reaction. EMBO Rep. 9:802-809.
22. Cheng, Y. and W. H. Prusoff. 1973. Relationship between the inhibition constant (K1) and the concentration of inhibitor which causes 50 percent inhibition (150) of an enzymatic reaction. Biochem. Pharmacol. 22:3099-3108.
23. Arnoldo, A., J. Curak, S. Kittanakom, I. Chevelev, V. T. Lee, M. Sahebol-Amri, B. Koscik, L. Ljuma, P. J. Roy, A. Bedalov, G. Giaever, C. Nislow, R. A. Merrill, S. Lory, and I. Stagljar. 2008. Identification of small molecule inhibitors of Pseudomonas aeruginosa exoenzyme S using a yeast phenotypic screen. PLoS. Genet. 4:e1000005.
24. Kabsch, W. 1993. Automatic Processing of Rotation Diffraction Data from Crystals of Initially Unknown Symmetry and Cell Constants. Journal of Applied Crystallography 26:795-800.
25. Vagin, A. and A. Teplyakov. 1997. MOLREP: an automated program for molecular replacement. Journal of Applied Crystallography 30:1022-1025.
26. McCoy, A. J., R. W. Grosse-Kunstleve, P. D. Adams, M. D. Winn, L. C. Storoni, and R. J. Read. 2007. Phaser crystallographic software. J. Appl. Crystallogr. 40:658-674.
27. Emsley, P. and K. Cowtan. 2004. Coot: model-building tools for molecular graphics. Acta Crystallogr. D. Biol. Crystallogr. 60:2126-2132.
28. Murshudov, G. N., A. A. Vagin, and E. J. Dodson. 1997. Refinement of macromolecular structures by the maximum-likelihood method. Acta Crystallogr. D. Biol. Crystallogr. 53:240-255.
29. Visschedyk, D. D., A. A. Perieteanu, Z. J. Turgeon, R. J. Fieldhouse, J. F. Dawson, and A. R. Merrill. 2010. Photox, a novel actin-targeting mono-ADP-ribosyltransferase from Photorhabdus luminescens. J. Biol. Chem. 285:13525-13534.
30. Scherrer, R., L. Louden, and P. Gerhardt. 1974. Porosity of the yeast cell wall and membrane. J. Bacteriol. 118:534-540.
31. Aouida, M., O. Tounekti, O. Belhadj, and L. M. Mir. 2003. Comparative roles of the cell wall and cell membrane in limiting uptake of xenobiotic molecules by Saccharomyces cerevisiae. Antimicrob. Agents Chemother. 47:2012-2014.
32. Kounnas, M. Z., R. E. Morris, M. R. Thompson, D. J. FitzGerald, D. K. Strickland, and C. B. Saelinger. 1992. The alpha 2-macroglobulin receptor/low density lipoprotein receptor-related protein binds and internalizes Pseudomonas exotoxin A. J. Biol. Chem. 267:12420-12423.
33. Du, X., R. J. Youle, D. J. FitzGerald, and I. Pastan. 2010. Pseudomonas Exotoxin A-mediated Apoptosis is Bak-dependent and Preceded by the Degradation of Mcl-1. Mol. Cell Biol.
34. McPherson, A. 2004. Introduction to protein crystallization. Methods 34:254-265.
35. Bell, C. E., T. O. Yeates, and D. Eisenberg. 1997. Unusual conformation of nicotinamide adenine dinucleotide (NAD) bound to diphtheria toxin: a comparison with NAD bound to the oxidoreductase enzymes. Protein Sci. 6:2084-2096.
36. Lipinski, C. A., F. Lombardo, B. W. Dominy, and P. J. Feeney. 2001. Experimental and computational approaches to estimate solubility and permeability in drug discovery and development settings. Adv. Drug Deliv. Rev. 46:3-26.
37. Jagtap, P., F. G. Soriano, L. Virag, L. Liaudet, J. Mabley, E. Szabo, G. Hasko, A. Marton, C. B. Lorigados, F. Gallyas, B. Sumegi, D. G. Hoyt, E. Baloglu, J. VanDuzer, A. L. Salzman, G. J. Southan, and C. Szabo. 2002. Novel phenanthridinone inhibitors of poly(adenosine 5′-diphosphateribose) synthetase: Potent cytoprotective and antishock agents. Critical Care Medicine 30:1071-1082.
38. Spudich, J. A., and Watt, S. 1971. J. Biol. Chem. 246:4866-4871.
39. Visschedyk, D. D., Perieteanu A. A., Turgeon Z. J., Fieldhouse R. J., Dawson J. F. and Merrill A. R. 2010. J. Biol. Chem. 285:13525-13534.

1. A method to inhibit bacterial virulence factors comprising administering an effective amount of a compound of Formula I: 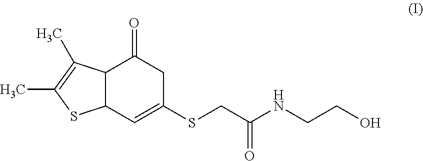
or a pharmaceutically acceptable solvate and/or prodrug thereof, to a subject or cell in need thereof.

2. A method to inhibit bacterial virulence factors comprising administering an effective amount of a compound of Formula II: 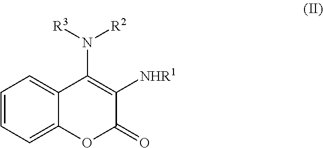
wherein:
R¹ is selected from H and C(O)C₁₋₄alkyl;
R² is H and R³ is selected from aryl and (CH₂)ₙNR⁴R⁵, or R² and R³, together with the nitrogen atom to which they are attached, form a piperidinyl, piperazinyl or morpholinyl ring;
R⁴ and R⁵ are independently selected from H, C₁₋₄alkyl or, together with the nitrogen atom to which they are attached, form a piperidinyl, piperazinyl or morpholinyl ring; and
n is 1, 2, 3 or 4,
or a pharmaceutically acceptable salt, solvate and/or prodrug thereof, to a subject or cell in need thereof.

3. The method of claim 2, wherein, in the compound of Formula II, R¹ is selected from H, C(O)CH₃ and C(O)CH₂CH₃.

4. The method of claim 2, wherein, in the compound of Formula II, R² is H and R³ is selected from phenyl and (CH₂)ₙNR⁴R⁵, wherein R⁴ and R⁵ are independently selected from H, CH₃ and CH₂CH₃ or, together with the nitrogen atom to which they are attached, form a piperidinyl, piperazinyl or morpholinyl ring and n is 2 or 3.

5. The method of claim 2, wherein, in the compound of Formula II, R² and R³, together with the nitrogen atom to which they are attached, form a piperidinyl, piperazinyl or morpholinyl ring.

6. The method of claim 2, wherein the compound of Formula II is selected from: 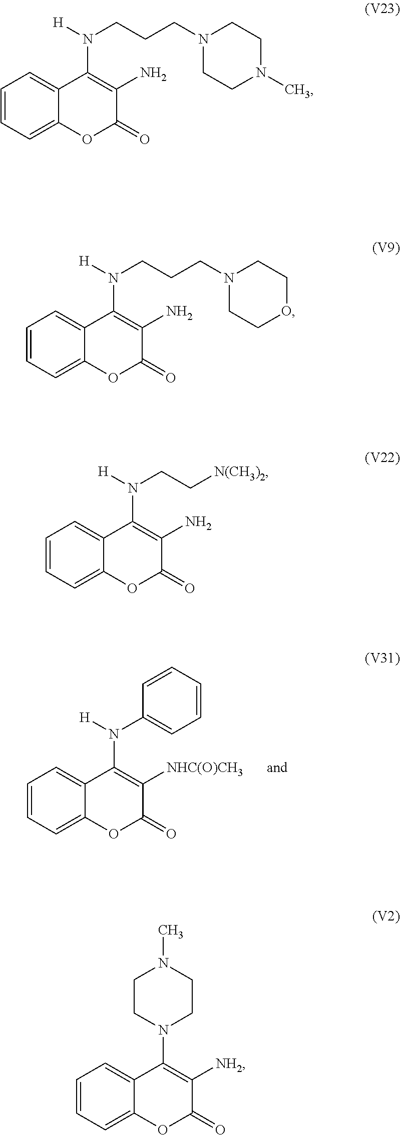
or a pharmaceutically acceptable salt, solvate and/or prodrug thereof.

7. The method of claim 6, wherein the compound of Formula II is 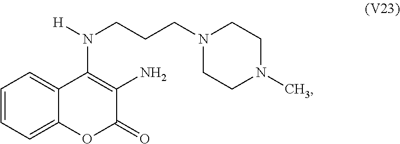
or a pharmaceutically acceptable salt, solvate and/or prodrug thereof.

8. The method of claim 1, wherein the bacterial virulence factor is Exo A.

9. The method of claim 2, wherein the bacterial virulence factor is Exo A.

10. A method treat or prevent a disease state caused by a bacterial infection comprising administering an effective amount of a compound of Formula I: 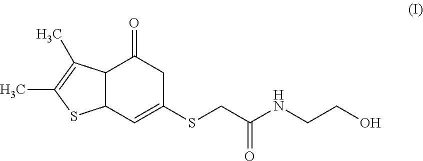
or a pharmaceutically acceptable solvate and/or prodrug thereof, to a subject or cell in need thereof.

11. A method to treat or prevent a disease state caused by a bacterial infection comprising administering an effective amount of a compound of Formula II: 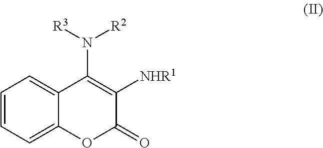
wherein:
R¹ is selected from H and C(O)C₁₋₄alkyl;
R² is H and R³ is selected from aryl and (CH₂)ₙNR⁴R⁵, or R² and R³, together with the nitrogen atom to which they are attached, form a piperidinyl, piperazinyl or morpholinyl ring;
R⁴ and R⁵ are independently selected from H, C₁₋₄alkyl or, together with the nitrogen atom to which they are attached, form a piperidinyl, piperazinyl or morpholinyl ring; and
n is 1, 2, 3 or 4,

12. The method of claim 11, wherein, in the compound of Formula II, R¹ is selected from H, C(O)CH₃ and C(O)CH₂CH₃.

13. The method of claim 11, wherein, in the compound of Formula II, R² is H and R³ is selected from phenyl and (CH₂)ₙNR⁴R⁵, wherein R⁴ and R⁵ are independently selected from H, CH₃ and CH₂CH₃ or, together with the nitrogen atom to which they are attached, form a piperidinyl, piperazinyl or morpholinyl ring and n is 2 or 3.

14. The method of claim 11, wherein, in the compound of Formula II, R² and R³, together with the nitrogen atom to which they are attached, form a piperidinyl, piperazinyl or morpholinyl ring.

15. The method of claim 11, wherein the compound of Formula II is selected from: 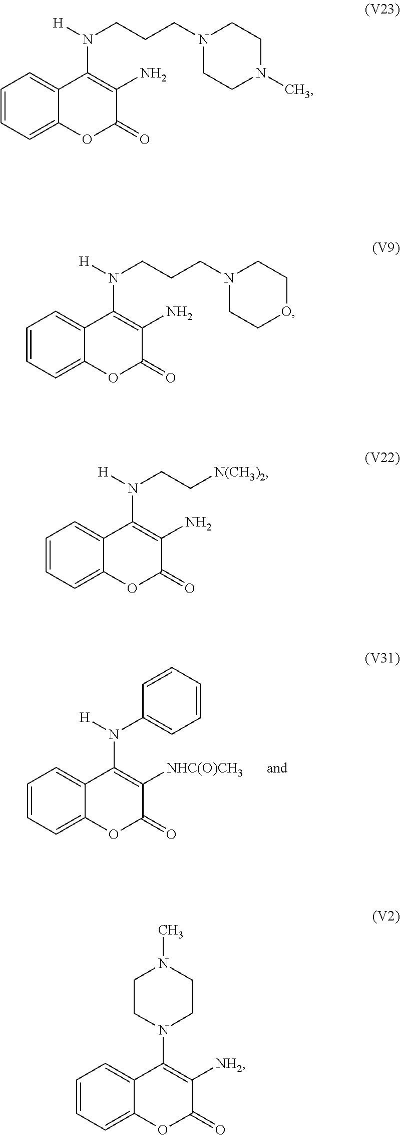
or a pharmaceutically acceptable salt, solvate and/or prodrug thereof.

16. The method of claim 11, wherein the compound of Formula II is 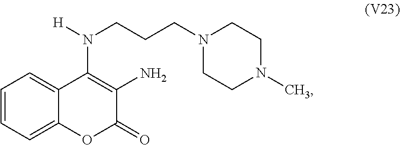
or a pharmaceutically acceptable salt, solvate and/or prodrug thereof.

17. The method of claim 10, wherein the bacterial infection is a P. aeruginosa infection.

18. The method of claim 11, wherein the bacterial infection is a P. aeruginosa infection.
